(19) United States
(12) Patent Application Publication
(10) Pub. No.: US 2026/0193309 A1
(43) Pub. Date: Jul. 9, 2026

(54) ENGINEERED VARIANTS OF HUMAN CEACAM1

(71) Applicant: The Brigham and Women’s Hospital, Inc., Boston, MA (US)

(72) Inventors: Richard S. Blumberg, Weston, MA (US); Amit Gandhi, Billerica, MA (US)

(21) Appl. No.: 19/100,052

(22) PCT Filed: Aug. 1, 2023
(86) PCT No.: PCT/US2023/071462

§ 371 Date: Jan. 30, 2025

Related U.S. Application Data
(60) Provisional application No. 63/370,008, filed on Aug. 1, 2022.

(51) Int. Cl. C07K 14/705; A61K 38/00; A61P 29/00; A61P 37/06

(52) U.S. Cl. CPC C07K 14/70503; A61P 29/00; A61P 37/06; A61K 38/00; C07K 2319/30

(57) ABSTRACT

Provided herein are high affinity dimer CEACAM1 variants. These variants enhance the hCEACAM1 mediated modulation of immunity in the treatment of infectious agents, autoimmunity, inflammation and cancer.

### CROSS REFERENCE TO RELATED APPLICATIONS

This application claims the benefit of priority under 35 U.S.C. § 119(e) to U.S. Provisional Application No. 63/370,008, filed Aug. 1, 2022. The entire contents of this application is incorporated herein by reference in its entirety.

### STATEMENT REGARDING FEDERALLY SPONSORED RESEARCH OR DEVELOPMENT

This invention was made with government support under DK51362 awarded by the National Institutes of Health. The government has certain rights in the invention.

### FIELD

The present invention relates generally to the field of molecular biology and medicine. More particularly, the invention provides CEACAM variants in the treatment of diseases such as, cancer, inflammatory bowel disease, autoimmune diseases.

### BACKGROUND

Carcinoembryonic antigen-related cell adhesion molecule 1 (CEACAM1) is a member of the carcinoembryonic antigen (CEA) family of immunoglobulin (Ig) like transmembrane glycoproteins. CEACAM family members are involved in cell-cell recognition and modulate cellular processes that range from the shaping of tissue architecture and neovascularization to the regulation of insulin homeostasis and T cell proliferation.

Various cellular activities have been attributed to the CEACAM1 protein, including roles in the differentiation and arrangement of tissue three-dimensional structure, angiogenesis, apoptosis, tumor suppression, metastasis, and the modulation of innate and adaptive immune responses. Further, several cell types potentially express CEACAM1, including tumor cells, T cells, B cells, natural killer (NK) cells, and certain macrophages.

### SUMMARY

Provided herein are engineered variants of human carcinoembryonic-antigen-related cell-adhesion molecule 1 (hCEACAM1). Methods of treating a subject include administration of one or more compositions embodied herein.

Accordingly, in certain aspects, a human carcinoembryonic-antigen-related cell-adhesion molecule 1 (hCEACAM1) protein of SEQ ID NO: 2, variant or functional fragment thereof, comprises one or more one or more amino acid substitutions comprising an R38D substitution, V39C substitution, a D40R substitution, a Q89H substitution, a G41S substitution, a V39F substitution, an I91F substitution, or combinations thereof. In certain embodiments, the hCEACAM1 protein, variant or functional fragment thereof, comprises an R38D substitution. In certain embodiments, the hCEACAM1 protein, variant or functional fragment thereof, comprises an V39C substitution. In certain embodiments, the hCEACAM1 protein, variant or functional fragment thereof, comprises a D40R substitution. In certain embodiments, the hCEACAM1 protein, variant or functional fragment thereof, comprises a Q89H substitution. In certain embodiments, the hCEACAM1 protein, variant or functional fragment thereof, comprises an G41S substitution. In certain embodiments, the hCEACAM1 protein, variant or functional fragment thereof, comprises an V39F substitution. In certain embodiments, the hCEACAM1 protein comprises, variant or functional fragment thereof, an I91F substitution. In certain embodiments, the hCEACAM1 protein, variant or functional fragment thereof, comprises an R38D substitution, a G41S substitution and a Q89H substitution. In certain embodiments, the hCEACAM1 protein, variant or functional fragment thereof, comprises a G41S substitution and a Q89H substitution. In certain embodiments, the hCEACAM1 protein, variant or functional fragment thereof, comprises a V39F substitution and an I91F substitution. In certain embodiments, the hCEACAM1 protein, variant or functional fragment thereof, comprises a V39F substitution and an G41S substitution. In certain embodiments, the hCEACAM1 protein, variant or functional fragment thereof, comprises a V39F substitution, a G41S substitution and a Q89H substitution. In certain embodiments, the hCEACAM1 protein, variant or functional fragment thereof, further comprises one or more moieties. In certain embodiments, the one or more moieties comprise an immunoglobulin Fc region, an Fc receptor, an antibody, antibody fragments, an aptamer, a detectable label, a ligand, a therapeutic agent, a cytotoxic agent, a receptor or fragments thereof. In certain embodiments, the hCEACAM1 protein, variant or functional fragment thereof is modified. In certain embodiments, the modification comprises glycosylation, deglycosylation, acetylation, phosphorylation, lipidation or combinations thereof. In certain embodiments, the hCEACAM1 protein, variant or functional fragment thereof, comprises one or more non-naturally-occurring amino acids.

In another aspect, a method of modulating an immune response for treating a disease or disorder in a subject comprising, administering to a subject a pharmaceutical composition comprising a therapeutically effective amount of a human carcinoembryonic-antigen-related cell-adhesion molecule 1 (hCEACAM1) protein of SEQ ID NO: 2, variant or functional fragment thereof, having one or more one or more amino acid substitutions comprising an R38D substitution, V39C substitution, a D40R substitution, a Q89H substitution, a G41S substitution, a V39F substitution, an I91F substitution, or combinations thereof, thereby, modulating the immune response of the subject. In certain embodiments, the subject is suffering from an autoimmune disease, cancer, an inflammatory disease, a bacterial infection, a viral infection or combinations thereof. In certain embodiments, the hCEACAM1 protein, variant or functional fragment thereof, comprises an R38D substitution. In certain embodiments, the hCEACAM1 protein, variant or functional fragment thereof, comprises an V39C substitution. In certain embodiments, the hCEACAM1 protein, variant or functional fragment thereof, comprises a D40R substitution. In certain embodiments, the hCEACAM1 protein, variant or functional fragment thereof, comprises a Q89H substitution. In certain embodiments, the hCEACAM1 the protein, variant or functional fragment thereof, comprises an G41S substitution. In certain embodiments, the hCEACAM1 protein, variant or functional fragment thereof, comprises an V39F substitution. In certain embodiments, the hCEACAM1 protein comprises, variant or functional fragment thereof, an I91F substitution. In certain embodiments, the hCEACAM1 protein, variant or functional fragment thereof, comprises an R38D substitution, a G41S substitution and a Q89H substitution. In certain embodiments, the hCEACAM1 protein, variant or functional fragment thereof, comprises a G41S substitution and a Q89H substitution. In certain embodiments, the hCEACAM1 protein, variant or functional fragment thereof, comprises a V39F substitution and an I91F substitution. In certain embodiments, the hCEACAM1 protein, variant or functional fragment thereof, comprises a V39F substitution and an G41S substitution. In certain embodiments, the hCEACAM1 protein, variant or functional fragment thereof, comprises a V39F substitution, a G41S substitution and a Q89H substitution. In certain embodiments, the hCEACAM1 protein, variant or functional fragment thereof, further comprises one or more moieties. In certain embodiments, the one or more moieties comprise an immunoglobulin Fc region, an antibody, antibody fragments, an aptamer, a detectable label, a ligand, a therapeutic agent, a cytotoxic agent, a receptor or fragments thereof. In certain embodiments, the hCEACAM1 protein, variant or functional fragment thereof is modified. In certain embodiments, the modification comprises glycosylation, deglycosylation, acetylation, phosphorylation, lipidation or combinations thereof. In certain embodiments, the hCEACAM1 protein, variant or functional fragment thereof, comprises one or more non-naturally-occurring amino acids.

In another aspect, an isolated nucleic acid encodes a human carcinoembryonic-antigen-related cell-adhesion molecule 1 (hCEACAM1) protein, variant or functional fragment thereof. In certain embodiments, the nucleic acid sequence encodes an hCEACAM1 sequence having at least a 90% sequence identity to any one of SEQ ID NOs: 1-4. In certain embodiments, the nucleic acid sequence encodes an hCEACAM1 peptide sequence comprising any one of SEQ ID NOs: 1-4. In certain embodiments, the nucleic acid sequence encodes an hCEACAM1 peptide sequence comprising SEQ ID NO: 1. In certain embodiments, the nucleic acid sequence encodes an hCEACAM1 peptide sequence comprising SEQ ID NO: 2. In certain embodiments, the nucleic acid sequence encodes an hCEACAM1 peptide sequence comprising SEQ ID NO: 3. In certain embodiments, the nucleic acid sequence encodes an hCEACAM1 peptide sequence comprising SEQ ID NO: 4.

In another aspect, an isolated nucleic acid encodes a human carcinoembryonic-antigen-related cell-adhesion molecule 1 (hCEACAM1) comprising an amino sequence having at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the nucleic acid sequence encodes an hCEACAM1 protein, variant or functional fragment thereof, comprising at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the nucleic acid sequence encodes an hCEACAM1 protein, variant or functional fragment thereof, comprising at least a 90%, 91%, 92%, 93%, 94%, 95%, 96%, 97%, 98%, or 99%, sequence identity to SEQ ID NO: 1. In certain embodiments, the nucleic acid sequence encodes an hCEACAM1 protein, variant or functional fragment thereof, comprising at least a 95%, 96%, 97%, 98%, or 99%, sequence identity to SEQ ID NO: 1.

In another aspect, an isolated nucleic acid encodes a human carcinoembryonic-antigen-related cell-adhesion molecule 1 (hCEACAM1) comprising an amino sequence having at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, the nucleic acid sequence encodes an hCEACAM1 protein, variant or functional fragment thereof, comprising at least a 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, the nucleic acid sequence encodes an hCEACAM1 protein, variant or functional fragment thereof, comprising at least a 90%, 91%, 92%, 93%, 94%, 95%, 96%, 97%, 98%, or 99%, sequence identity to SEQ ID NO: 2. In certain embodiments, the nucleic acid sequence encodes an hCEACAM1 protein, variant or functional fragment thereof, comprising at least a 95%, 96%, 97%, 98%, or 99%, sequence identity to SEQ ID NO: 2.

In another aspect, an isolated nucleic acid encodes an amino sequence having at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 3. In certain embodiments, the nucleic acid sequence encodes an amino sequence comprising at least a 75%, 80%, 85%, 86%, 87%, 88%, 89%, 90%, 91%, 92%, 93%, 94%, 95%, 96%, 97%, 98%, or 99%, sequence identity to SEQ ID NO: 3. In certain embodiments, the nucleic acid sequence encodes an amino sequence comprising at least a 90%, 91%, 92%, 93%, 94%, 95%, 96%, 97%, 98%, or 99%, sequence identity to SEQ ID NO: 3. In certain embodiments, the nucleic acid sequence encodes an amino sequence comprising at least a 95%, 96%, 97%, 98%, or 99%, sequence identity to SEQ ID NO: 3.

In another aspect, an isolated nucleic acid encodes an amino sequence having at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 4. In certain embodiments, the nucleic acid sequence encodes an amino sequence comprising at least a 75%, 80%, 85%, 86%, 87%, 88%, 89%, 90%, 91%, 92%, 93%, 94%, 95%, 96%, 97%, 98%, or 99%, sequence identity to SEQ ID NO: 4. In certain embodiments, the nucleic acid sequence encodes an amino sequence comprising at least a 90%, 91%, 92%, 93%, 94%, 95%, 96%, 97%, 98%, or 99%, sequence identity to SEQ ID NO: 4. In certain embodiments, the nucleic acid sequence encodes an amino sequence comprising at least a 95%, 96%, 97%, 98%, or 99%, sequence identity to SEQ ID NO: 4.

In another aspect, an isolated nucleic acid encodes a human carcinoembryonic-antigen-related cell-adhesion molecule 1 (hCEACAM1) protein, variant or functional fragment of SEQ ID NO: 2, having one or more one or more amino acid substitutions comprising an R38D substitution, V39C substitution, a D40R substitution, a Q89H substitution, a G41S substitution, a V39F substitution, an I91F substitution, or combinations thereof. In certain embodiments, the encoded protein, variant or functional fragment thereof, comprises an R38D substitution. In certain embodiments, the encoded protein, variant or functional fragment thereof, comprises an V39C substitution. In certain embodiments, the encoded protein, variant or functional fragment thereof, comprises a D40R substitution. In certain embodiments, the encoded protein, variant or functional fragment thereof, comprises a Q89H substitution. In certain embodiments, the encoded protein, variant or functional fragment thereof, comprises an G41S substitution. In certain embodiments, the encoded protein, variant or functional fragment thereof, comprises an V39F substitution. In certain embodiments, the encoded protein comprises, variant or functional fragment thereof, an 191F substitution. In certain embodiments, the encoded protein, variant or functional fragment thereof, comprises an R38D substitution, a G41S substitution and a Q89H substitution. In certain embodiments, the encoded protein, variant or functional fragment thereof, comprises a G41S substitution and a Q89H substitution. In certain embodiments, the encoded protein, variant or functional fragment thereof, comprises a V39F substitution and an I91F substitution. In certain embodiments, the encoded protein, variant or functional fragment thereof, comprises a V39F substitution and an G41S substitution. In certain embodiments, the encoded protein, variant or functional fragment thereof, comprises a V39F substitution, a G41S substitution and a Q89H substitution.

In another aspect, a vector comprises an isolated nucleic acid encoding a protein embodied herein.

In another aspect, an isolated cell comprises a vector encoding an isolated nucleic acid encoding a protein embodied herein.

### Definitions

The terminology used herein is for the purpose of describing particular embodiments only and is not intended to be limiting of the disclosure. Unless specifically defined otherwise, all technical and scientific terms used herein shall be taken to have the same meaning as commonly understood by one of ordinary skill in the art (e.g., crystallography, in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about. Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

As used herein, the terms “agent” or “moiety” are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, hormones, oligonucleotides, enzymes, peptides organic or inorganic molecules, natural or synthetic compounds and the like. An agent can be assayed in accordance with the methods of the disclosure at any stage during clinical trials, during pre-trial testing, or following FDA-approval.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The term “amino acid” as used herein refers to naturally occurring and synthetic or non-naturally occurring α, β, γ, and δ amino acids, and includes but is not limited to, amino acids found in proteins, i.e., glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. Alternatively, the amino acid can be a derivative of alanyl, valinyl, leucinyl, isoleucinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleucinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl. When the term amino acid is used, it is considered to be a specific and independent disclosure of each of the esters of α, β, γ, and δ glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine in the D and L-configurations. Examples of non-naturally occurring amino acids include, but are not limited to, D-amino acids (i.e., an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (NIe), norvaline (Nva), homoarginine (Har), 4-aminobutyric acid (γ-Abu), 2-aminoisobutyric acid (Aib), 6-aminohexanoic acid (ε-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

The term “amino acid sequence” is the order in which amino acid residues, connected by peptide bonds, lie in the chain in peptides and proteins.

The transitional term “comprising,” which is synonymous with “including,” “having”, “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed disclosure.

As used herein, the term “cellular immune response” can be used interchangeably with the term “cell-mediated immune response” and refers to an immune response that does not predominantly involve antibodies. Instead, a cellular immune response involves the activation of different immune cells (e.g., phagocytes and antigen-specific cytotoxic T-lymphocytes) that produce various effector molecules (e.g., cytokines, perforin, granzymes) upon activation (e.g., via antigen stimulation). As used herein, the term “humoral immune response” refers to an immune response predominantly mediated by macromolecules found in extracellular fluids, such as secreted antibodies, complement proteins, and certain antimicrobial peptides. The term “antibody-mediated immune response” refers to an aspect of a humoral immune response that is mediated by antibodies.

The term “combination therapy”, as used herein, refers to those situations in which two or more different pharmaceutical agents (e.g., an hCEACAM1 or variants thereof and a chemotherapeutic agent) are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the terms “conjugated,” “linked,” “attached,” “fused” and “tethered,” when used with respect to two or more moieties, means that the moieties or domains are physically associated or connected with one another, either directly or via one or more additional moieties that serve as a linking agent, to form a structure that is sufficiently stable so that the moieties remain physically associated under the conditions in which the structure is used, e.g., physiological conditions. The linkage can be based on genetic fusion according to the methods known in the art or can be performed by, e.g., chemical cross-linking. The compounds and targeting agents may be linked by a flexible linker, such as a polypeptide linker. The polypeptide linker can comprise plural, hydrophilic or peptide-bonded amino acids of varying lengths. The term “associated” will be used for the sake of brevity and is meant to include all possible methods of physically associating each compound to a targeting ligand.

A “detectable label” or a “label” is a composition detectable by spectroscopic, photochemical, biochemical, immunochemical, or chemical means. For example, useful labels include radio labeled molecules fluorophores, radiochemical, luminescent compounds, electron-dense reagents, enzymes (e.g., as commonly used in an ELISA), biotin, radioactive compounds, non-radioactive compounds, digoxigenin, or haptens and proteins which can be made detectable, e.g., by incorporating a label into the peptide.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

By “encoding” or “encoded”, “encodes”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Suitable native-sequence Fc regions of the disclosure include human IgG1, IgG2 (IgG2A, IgG2B), IgG3 and IgG4.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (See, M. Daeron, Annu. Rev. Immunol. 15:203-234 (1997). FcRs are reviewed in Ravetch and Kinet, Annu. Rev. Immunol. 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus. Guyer et al., J. Immunol. 117:587 (1976) and Kim et al., J. Immunol. 24:249 (1994). Methods of measuring binding to FcRn are known (see, e.g., Ghetie and Ward, Immunol. Today 18: (12): 592-8 (1997); Ghetie et al., Nature Biotechnology 15 (7): 637-40 (1997); Hinton et al., J. Biol. Chem. 279 (8): 6213-6 (2004); WO 2004/92219 (Hinton et al.). Binding to FcRn in vivo and serum half-life of human FcRn high-affinity binding polypeptides can be assayed, e.g., in transgenic mice or transfected human cell lines expressing human FcRn, or in primates to which the polypeptides having a variant Fc region are administered. WO 2004/42072 (Presta) describes antibody variants which improved or diminished binding to FcRs. See also, e.g., Shields et al., J. Biol. Chem. 9 (2): 6591-6604 (2001).

As used herein, the term “immune cells” refers to any cells of the immune system that are involved in mediating an immune response. Non-limiting examples of immune cells include a T lymphocyte, B lymphocyte, natural killer (NK) cell, macrophage, eosinophil, mast cell, dendritic cell, neutrophil, or combination thereof. In some aspects, an immune cell expresses CD3. In certain aspects, the CD3-expressing immune cells are T cells (e.g., CD4⁺ T cells or CD8⁺ T cells). In some aspects, an immune cell that can be targeted with a targeting moiety (e.g., anti-CD3) comprises a naive CD4⁺ T cell. In some aspects, an immune cell comprises a memory CD4⁺ T cell. In some aspects, an immune cell comprises an effector CD4⁺ T cell. In some aspects, an immune cell comprises a naïve CD8″ T cell. In some aspects, an immune cell comprises a memory CD8⁺ T cell. In some aspects, an immune cell comprises an effector CD8⁺ T cell. In some aspects, an immune cell is a dendritic cell. In certain aspects, a dendritic cell comprises a plasmacytoid dendritic cell (pDC), a conventional dendritic cell 1 (cDC1), a conventional dendritic cell 2 (cDC2), inflammatory monocyte derived dendritic cells, Langerhans cells, dermal dendritic cells, lysozyme-expressing dendritic cells (LysoDCs), Kupffer cells, or any combination thereof.

An “immune response,” as used herein, refers to a biological response within a vertebrate against foreign agents, e.g., virus, or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell. Accordingly, an immune response can comprise a humoral immune response (e.g., mediated by B-cells), cellular immune response (e.g., mediated by T cells), or both humoral and cellular immune responses. In some aspects, an immune response is an “inhibitory” immune response. An “inhibitory” immune response is an immune response that blocks or diminishes the effects of a stimulus (e.g., antigen). In certain aspects, the inhibitory immune response comprises the production of inhibitory antibodies against the stimulus. In some aspects, an immune response is a “stimulatory” immune response. A “stimulatory” immune response is an immune response that results in the generation of effectors cells (e.g., cytotoxic T lymphocytes) that can destroy and clear a target antigen (e.g., tumor antigen or viruses).

By the term “modulate,” it is meant that any of the mentioned activities, are, e.g., increased, enhanced, increased, agonized (acts as an agonist); or, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values. Modulation can also normalize an activity to a baseline value.

The terms “subject”, “patient” or “individual” are used interchangeably herein, and refers to a mammalian subject to be treated, with human patients being preferred. In some cases, the methods of the disclosure find use in experimental animals, in veterinary application, and in the development of animal models for disease, including, but not limited to, rodents including mice, rats, and hamsters; and primates. Patients in need of therapy comprise those at risk of developing a certain condition, disease or disorder (e.g. due to genetic, environmental or physical attributes, such as for example, obesity). Patients in need of therapy also include those afflicted with a condition, disease or disorder. The diseases or disorders comprise, for example: autoimmune diseases, cancer, inflammatory diseases, neurological diseases or disorders, neuroinflammatory diseases or disorders, cardiovascular disease, diseases or disorders caused by infectious agents such as, for example, viruses, bacteria, fungi, prions, or parasites.

The terms “therapeutically effective amount,” “treatment effective amount” and “effective amount” as used herein are synonymous unless otherwise indicated, and mean an amount of a compound, peptide or composition of the present disclosure that is sufficient to improve the condition, disease, or disorder being treated and/or achieved the desired benefit or goal. Those skilled in the art will appreciate that the therapeutic effects need not be complete or curative, as long as some benefit is provided to the subject. Determination of a therapeutically effective amount, as well as other factors related to effective administration of a compound of the present disclosure to a subject of this disclosure, including dosage forms, routes of administration, and frequency of dosing, may depend upon the particulars of the condition that is encountered, including the subject and condition being treated or addressed, the severity of the condition in a particular subject, the particular compound being employed, the particular route of administration being employed, the frequency of dosing, and the particular formulation being employed. Determination of a therapeutically effective treatment regimen for a subject of this disclosure is within the level of ordinary skill in the medical or veterinarian arts. In clinical use, an effective amount may be the amount that is recommended by the U.S. Food and Drug Administration, or an equivalent foreign agency. The amount of active ingredient that can be combined with the carrier materials to produce a single dosage form varies depending upon the subject being treated and the particular mode of administration.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Any compositions or methods provided herein can be combined with one or more of any of the other compositions and methods provided herein.

### BRIEF DESCRIPTION OF THE DRAWINGS

FIGS. 1A, 1B, and 1C show the crystal structure of a hCEACAM1 oligomer (PDB code 7RPP resolution (2.20 Å) involving GC′ and ABED interfaces as well as GFCC′ face-mediated hydrogen-bond and hydrophobic interactions as observed in hCEACAM1 oligomer crystal structure (PDB code 7RPP) and comparison with WT homodimer (PDB code 4QXW). FIG. 1A. Ribbon diagram of the hCEACAM1 IgV domain, whereas hCEACAM1 molecule b and molecule c make GFCC′ face interactions and molecule a and molecule b make ABED face interactions. FIG. 1B. Surface representation for molecule a, b, and c respectively, which are involved in simultaneous GFCC′ (856.2 Å2) and ABED (482.1 Å2) face interactions (shown by black circles). FIG. 1C. Ribbon diagram of the hCEACAM1 IgV domain with GFCC′ and ABED face interactions. The left inset shows ABED face interactions between residues of molecules a and molecule b, wherein residues Q54, P59, G63 167, Q76 of molecule a make six hydrogen-bond interactions with residues N23, Q26, Q27, Q53, S93 of molecule b. The right inset shows GFCC′ face interactions between residues of molecules b and molecule c, wherein residues F29, Y34, V39, Q89, I91, and N97 of molecules b and c make various hydrogen-bond and hydrophobic interactions with each other. The residues of molecule a and molecule c are shown in italics in left and right insets, respectively. FIG. 1D. Fifteen (15) hydrogen-bond interactions through GFCC′ face residues as observed in the crystal structure between molecule b (left) and molecule c (right) residues. The hydrogen-bond interactions (15) across the GFCC′ face by labelled residues described are shown by dashed lines. The asterisk (*) indicates two hydrogen-bonds (shown by single dashed line) mediated by Q89 residues of molecules b and c with each other via OE1 and NE2 atoms. Similar hydrogen-bond interactions were also observed in previously reported crystal structure of hCEACAM1 IgV domain (PDB code 4QXW) with exceptions of E37-R38 and N97A symmetrical interactions observed here. FIG. 1E. Hydrophobic interactions at GFCC′ face through F29, I91 and V39 residues are shown by arc/point representations. The molecule (a) and (b) residues are labeled with hydrophobic interactions as measured by distance between β carbons of labeled residues of molecules b and c. Similar hydrophobic interactions were also observed in previously reported crystal structure of hCEACAM1 Ig-V domain (PDB code 4QXW). FIG. 1F. Comparison of hydrogen-bond interactions through GFCC′ faces of previously reported dimer (PDB code 4QXW) and oligomer (PDB code 7RPP). Seventeen (17) hydrogen-bond interactions through GFCC′ face residues are observed in the WT homodimer crystal structure (PDB code 4QXW) between molecule a and molecule b (underlined) residues. The 17 hydrogen-bond interactions across the GFCC′ face by labelled residues described are shown by dashed lines. The interactions shown by solid lines were not observed in hCEACAM1 oligomeric crystal structure. The interactions shown by dashed dot lines between E37-R38 residues were observed only in oligomeric crystal structure, and not observed in homodimer crystal structure (PDB code 4QXW). The asterisk (*) indicates two hydrogen-bonds (shown by single dashed line) mediated by Q89 residues of molecules a and b with each other via OE1 and NE2 atoms. FIG. 1G. Ribbon diagram of the hCEACAM1 crystal structure reported here with GFCC′ face, where additional interactions between E37 and R³⁸ residues of molecules b (left) and c (right) were observed compared to previously reported crystal structure of hCEACAM1 IgV domain (PDB code 4QXW). E37 and R38 residues are shown by sticks. FIG. 1H. CEACAM1 residue 38 is not engaged in any interactions with other residues in the WT homodimer structure (PDB ID: 4QXW). Color versions of FIGS. 1A, 1B, and 1C can be found as FIGS. 1A, 1B, and 1C in Gandhi et al. Structural analysis of human CEACAM1 oligomerization. Commun Biol. 2022 Sep. 30; 5 (1): 1042, which is incorporated herein in its entirety. Color versions of FIGS. 1D, 1E, IF, and 1G can be found as Supplementary FIGS. 2A, 2B, 2C, and 2D in Gandhi et al. Structural analysis of human CEACAM1 oligomerization. Commun Biol. 2022 Sep. 30; 5 (1): 1042.

FIGS. 2A, 2B, and 2C show a glycosylated model of the hCEACAM1. FIG. 2A. Modeling of the N-linked sugars N-acetylglucosamine (NAG) and β-d-Mannose (BMA) onto the N70, N77, and N81 residues of all three human CEACAM1 molecules observed in the crystal structure, wherein hCEACAM1 molecules are shown by ribbon diagram. Right inset shows structure of two N-acetylglucosamine (NAG) and one β-d-Mannose (BMA) by stick representation, which are modeled onto each set of N70, N77, and N81 residues in the crystal structure shown in left and abbreviated as NNB in the figure. FIG. 2B. Modeled N-linked sugars N-acetylglucosamine (NAG) and β-d-Mannose (BMA) onto the N70, N77, and N81 residues of human CEACAM1 molecules (a, b) to not appear to block any of the ABED face hydrogen-bond interactions shown. FIG. 2C. Surface representation for molecules a, b, and c, respectively, and stick representation of modeled sugar molecules. GFCC′ and ABED face interactions are depicted by black oblong and three modeled NNB sugars on each hCEACAM1 molecule residues N70, N77 and N81 do not appear to block ABED and GFCC′ face interactions. hCEACAM1 molecules are labeled as above. Color versions of FIGS. 2A, 2B, and 2C can be found as FIGS. 2A, 2B, and 2C in Gandhi et al. Structural analysis of human CEACAM1 oligomerization. Commun Biol. 2022 Sep. 30; 5 (1): 1042.

FIGS. 3A and 3B show ¹⁵N-HSQC spectra of WT hCEACAM1 IgV domain binding with octyl beta-D-glucopyranoside (BOG). FIG. 3A. Wild type hCEACAM1 IgV domain (50 μM) binding with 10 mM octyl beta-D-glucopyranoside (BOG). An expanded region of overlaid 15N-HSQC spectra of hCEACAM1 WT alone and with BOG reveal spectral changes for a few residues. Peaks shift observed for residues V17, L18 and L73 are shown by arrow. FIG. 3B. No major peak shift changes (in ppm) were observed at the GFCC′ and ABED interface upon binding of BOG with the hCEACAM1 IgV domain with only a few shifted peaks from residues that localized to the ABED face consistent with binding of BOG near a hydrophobic patch of residues L73 and V17 as observed in the crystal structures (PDB codes 4QXW, 6XNT). Color versions of FIGS. 3A and 3B can be found as FIGS. 3A and 3C in Gandhi et al. Structural analysis of human CEACAM1 oligomerization. Commun Biol. 2022 Sep. 30; 5 (1): 1042.

FIGS. 4A and 4B show that human CEACAM1 higher-order oligomer formation is mediated by GFCC′ face. FIG. 4A. Human CEACAM1 higher-order oligomer formed by GFCC′ face-mediated interactions between two hCEACAM1 oligomers as observed in the crystal structure. Human CEACAM1 oligomers present in the crystal asymmetric unit and symmetry-related oligomer are shown, wherein molecules a, b and c and its symmetry mates labeled aₛ, bₛ, cₛ are shown by ribbon diagram. Central to higher-order oligomer formation is GFCC′ facemediated interactions between molecules a from the first trimer (formed by molecules a, b and c) and its symmetry-mate as the second trimer (formed by molecules ₐₛ, bₛ, and cₛ). FIG. 4B. Superimposition of the GFCC′-face mediated dimer present in both oligomers revealed similar GFCC-face mediated interactions, wherein GFCC′ face dimers formed by molecules b and c, and by molecules and it's symmetry-mate as are shown by ribbon diagram. Color versions of FIGS. 4A and 4B can be found as FIGS. 4A and 4B in Gandhi et al. Structural analysis of human CEACAM1 oligomerization. Commun Biol. 2022 Sep. 30; 5 (1): 1042.

FIG. 5 shows the crystallographic Debye-Waller factor (temperature factor or B factor) assignment of the crystal structure of the hCEACAM1 IgV domain with GFCC′ and ABED face interactions. The ribbon diagram of the hCEACAM1 molecules (a, b, and c), glycosylation sites (N70, N77 and N81 residues shown by spheres), and interacting residues at ABED face (shown by sticks) with B-factor assignment. The loops, a helices, β strand interacting ABED face residues and glycosylation sites are shown based on B-factor range (minimum=10, maximum=20). B-factor assignments revealed higher thermal motion or dynamic mobility in the side-chains in the some of the residues including Q27, Q53, Q54 at the ABED face and glycosylation site residues (N70, N77, and N81).

FIGS. 6A, 6B, 6C, and 6D show the results from UV-spectroscopy studies of hCEACAM1 IgV domain with various metal ions. FIG. 6A. Titration of hCEACAM1 WT 100 microM (with buffer) and with various metals (Ni⁺⁺, Zn⁺⁺, Mn⁺⁺, Li⁺⁺, Ca⁺⁺) at 1:1 and 1:2.5 ratio. OD analysis at 340 nm revealed Zn⁺⁺ or Ni⁺⁺ caused concentration dependent aggregation of the hCEACAM1 WT protein. FIG. 6B. OD analysis of supernatant after centrifugation for 2 min at 10000 rpm at 280 nm for WT protein and with various metals (Ni⁺⁺, Zn⁺⁺, Mn⁺⁺, Li⁺⁺, Ca⁺⁺) at 1:1 and 1:2.5 ratio. FIG. 6C. Titration of H105A mutant 100 μM (with buffer) and with various metals (Ni⁺⁺, Zn⁺⁺, Mn⁺⁺, Li⁺⁺, Ca⁺⁺) at 1:1 and 1:2.5 ratio. OD analysis at 340 nm revealed no Zn⁺⁺ or Ni⁺⁺ mediated aggregation observed for the H105A mutant protein. FIG. 6D. OD analysis of H105A mutant supernatant after centrifugation at 280 nm alone and with various metals (Ni⁺⁺, Zn⁺⁺, Mn⁺⁺, Li⁺, Ca⁺⁺) at 1:1 and 1:2.5 ratio. The mean values with standard deviations are shown in bar graph with error bars of the triplicate samples.

FIGS. 7A, 7B, and 7C show 15N-HSQC spectra of WT hCEACAM1 IgV domain with zinc chloride (ZnCl₂). 15N-HSQC spectra of 190 microM 15N-labeled WT hCEACAM1 IgV domain alone (FIG. 7A), and in the presence of 250 microM ZnCl2 (FIG. 7B, weakened peaks in black), and both 250 microM ZnCl₂ and 1 mM EDTA (FIG. 7C, recovered peaks in black). The weakened and missing backbone amide peaks caused by zinc bridging as shown in middle b panel are recovered after addition of EDTA (right c panel) to sequester zinc ions. Color versions of FIGS. 7A, 7B, and 7C can be found as FIGS. 7A, 7B, and 7C in Gandhi et al. Structural analysis of human CEACAM1 oligomerization. Commun Biol. 2022 Sep. 30; 5 (1): 1042.

FIGS. 8A, 8B, and 8C show 15N-HSQC spectra of hCEACAM1 WT IgV with nickel chloride (NiCl₂) and effect of EDTA. 15N-HSQC spectra of 190 microM 15N-labeled WT hCEACAM1 IgV domain alone (FIG. 8A), and in the presence of 250 microM NiCl₂ (FIG. 8B, weakened peaks in black), and both 250 microM NiCl2 and 1 mM EDTA (FIG. 8C, recovered peaks in black). The weakened and missing backbone amide peaks caused by nickel bridging are recovered after addition of EDTA to sequester nickel ions. Color versions of FIGS. 8A, 8B, and 8C can be found as FIGS. 7A, 7D, and 7E in Gandhi et al. Structural analysis of human CEACAM1 oligomerization. Commun Biol. 2022 Sep. 30; 5 (1): 1042.

FIG. 9 shows an oligomerization model of human CEACAM1. Human CEACAM1 dimerization is mediated by primary GFCC′ face (top left panel) which enables oligomerization through flexible ABED face (top right panel) as observed in the hCEACAM1 oligomeric crystal structure for molecules a, b, and c. The GFCC′ face is a central force of this oligomeric assembly as it not only governs human CEACAM1 monomer-dimer equilibrium and interactions with ligands, but also enables highly flexible ABED-mediated interactions to form oligomers (top right panel, wherein molecules b and c mediate GFCC′ face interactions and molecules a and b mediate ABED face interactions) and higher-order oligomers (bottom right panel showing two hCEACAM1 oligomers as observed in the crystal structure, formed by molecules a, b and c and its symmetry mates labeled aₛ, bₛ, cₛ, wherein molecules a and as mediate similar GFCC′ face interactions observed for molecules b and c). Having flexibility in GFCC′ and ABED face enables hCEACAM1 molecules to further bridge to other CEACAM1 molecules (shown here on bottom left panel by ribbon representation) through metal ions (Ni⁺⁺ or Zn⁺⁺, shown by orange sphere) via residues H105 as shown by green stick representation. A color version of FIG. 9 can be found as FIG. 8 in Gandhi et al. Structural analysis of human CEACAM1 oligomerization. Commun Biol. 2022 Sep. 30; 5 (1): 1042.

FIGS. 10A-10D: hCEACAM1 GFCC′ face high dimer is observed upon binding of R38D & R38D/G41S variants with WT type. FIG. 10A: hydrogen and salt-bridge interactions between WT-R38 and variant-D38. FIG. 10B: nineteen hydrogen bonds at GFCC′ face in R38D variant binding with WT as observed by PDB-PISA. FIG. 10C: hydrogen bonds and salt-bridge interactions between WT-R38 and variant-D38, and hydrogen bonds between WT-E99 and variant-Ser41. FIG. 10D: twenty hydrogen bonds at GFCC′ face in R38D/G41S variant binding with WT as observed by PDB-PISA.

FIG. 11 is a crystal structure of CEACAM1 Q89H variant, showing formation of high affinity dimer at GFCC′ face.

FIG. 12 demonstrates the higher binding of tagless R38D and Q89H compared to WT as observed in ELISA binding assays. **, P<0.01.

FIG. 13 is a crystal structure of D40R variant, showing D40R residue does not make interactions, but is predicted to bind with D40 of WT and forms a high affinity dimer.

FIGS. 14A-14C show the biophysical characterization of CEACAM1 IgV mutants.

Thermal stability and molecular size analysis of hCEACAM1 WT and GFCC′ face mutants.

FIG. 14A: The arc/stick representation of hydrophobic interactions by F29, I91, and V39 residues of molecules as observed in the E99A crystal structure compared to the WT. The hydrophobic interactions as measured by distance between β carbons of labeled residues are shown in for E99A mutant and WT, respectively. The weaker hydrophobic interactions mediated between two V39 residues are shown by fewer pointers on the hydrophobic arc relative to WT. FIG. 14B: Variations in melting point temperature (TM) determined by differential scanning fluorimetry (DSF) are shown for WT and mutant hCEACAM IgV. FIG. 14C: Size exclusion chromatography and multi-angle light scattering (SEC-MALS) differential refractive index (dRI) chromatograms and calculated molecular weights are displayed for WT (black), V39A, I91A, N97A, and E99A.

FIGS. 15A and 15B demonstrate that CEACAM1's tolerance-inducing functions can be engaged by in vivo administration of CEACAM1-Fc fusion proteins to reverse inflammation associated with experimental colitis. Mice that were induced to exhibit oxazolone-induced (hapten-mediated) colitis were treated with a mouse CEACAM1 N-domain as a fusion protein with human IgG1 Fc or human IgG1 Fc alone. After treatment, pathology (FIG. 15A) and the secretion of T cell derived cytokines from lamina propria lymphocytes (FIG. 15B) were determined. Note the severe inflammation in the control-Fc group with nearly complete loss of intestinal epithelial cells (IEC) and expansion of the lamina propria and the preservation of intestinal epithelial cells and decreased lamina propria expansion (in the presence of the CEACAM1-Fc fusion protein (FIG. 15A). The lamina propria T cells also secreted significantly lower levels of interferon-γ (IFNγ), an inflammatory protein, upon treatment with the CEACAM1-Fc fusion protein (FIG. 15B).

FIG. 16 demonstrates that ligation provided by a mouse CEACAM1 N-domain Fc fusion protein can inhibit T cell activation. Primary mouse splenic CD4 T cells (0.5×10⁶ cells/ml) were incubated in solution with soluble anti-CD3 (clone 145-2C11, 1 mg/ml) with or without anti-CD28 (clone 37.51, 2 mg/ml) in the presence of mouse CEACAM1 N domain—human IgG1 Fc fusion protein (5 mg/ml) or human IgG1 Fc (5 mg/ml) as a control for 96 hours at which time proliferation (based upon tritiated-thymidine, 3H, uptake as counts per minute or C.P.M.) and secretion of interferon-γ (IFNγ) and IL-2 were assessed. Note the dramatic inhibition of CD4⁺T cell proliferation and inflammatory cytokine secretion upon treatment with the CEACAM1-Fc fusion protein.

FIG. 17 demonstrates that CEACAM1 as an Fc-fusion protein can inhibit the activation of primary human T cells. Human peripheral blood mononuclear cells were cultured for 48 hours with phytohemagglutinin (PHA, a T cell mitogen). Proliferation was assessed in the presence or absence of either a human CEACAM1 N-domain containing-human IgG1 Fc fusion protein (CEACAM1-Ig) or CTLA4-Ig (human CTLA4 as a fusion protein with human Fc) at various concentrations. On day 3 of the stimulus, the cells were pulsed with BrdU for the last 16 hours of the assay. Positive (Pos) control is PHA stimulation alone. Negative (neg) is unstimulated cells. *, P<0.05 vs pos control. Note the significant inhibition of T cell proliferation in the presence of CEACAM1-Ig.

### DETAILED DESCRIPTION

The carcinoembryonic antigen-related adhesion molecules (CEACAM) are a subgroup of the CEA family, containing 12 human CEACAM genes including CEACAM1, CEACAM3-CEACAM8, CEACAM16 and CEACAM18-21. CEACAM family members are expressed on different cell types including epithelial cells, neutrophils, T cells etc. CEACAMs are heavily glycosylated glycoproteins attached to the plasma membrane of cells where they can act as intercellular adhesions molecules involved in a number of different processes including cell adhesion, proliferation, differentiation and tumor suppression. CEACAMs can also serve as bacterial adhesion molecules for, e.g., Escherichia coli, Neisseria spp, Salmonella spp and others, indicating they can play a role in mounting innate immune responses against microbes. Several CEACAMs including CEACAM1, CEACAM5, CEACAM7 are expressed in the luminal intestinal epithelial cells. Upon intestinal inflammation such as IBD, CEACAMs are altered. For example, CEACAM6 is highly increased in patients with CD and has been shown to serve as a receptor to CD-associated bacteria. Other CEACAMs such as CEACAM1, normally found on epithelial cells of the gut, is highly reduced in colon cancer.

### CEACAM1

Human (h) carcinoembryonic antigen-related cell adhesion molecule 1 (CEACAM1) is a type I glycosylated immunoglobulin (Ig)-related transmembrane protein that plays an important role in physiological processes associated with a variety of hematopoietic and stromal populations. In addition to intestinal epithelial cells (IECs), it is expressed on a wide range of cell types such as lymphocytes, endothelial cells and myeloid cells. In lymphocytes it is involved in T cell tolerance.

hCEACAM1 is characterized by 12 structural isoforms that are generated by alternative splicing and include a common N-terminal immunoglobulin-like (IgV) domain, by up to three of type 2 constant immunoglobulin (IgC2) ectodomains, and a transmembrane region that is coupled to either a short(S) or long (L) cytoplasmic tail important for signaling and inhibitory function in the case of L-isoforms which contain immunoreceptor tyrosine-based inhibitory motifs. The CEACAM1-L and CEACAM1-S isoforms can assemble as independent signaling units wherein they provide inhibitory and activating signals, respectively. Two isoforms (CEACAM1-3, CEACAM1-3C2) have premature start codons resulting in unique secreted isoforms. Like other hCEACAM family members, the hCEACAM1 IgV domain serves as the major ligand binding surface and is characterized by the presence of two anti-parallel β-sheets creating two distinct faces, the GFCC′ and ABED faces, respectively. In addition, IgV-like N-domain containing secreted isoforms also exist.

The sequence of the full-length form of CEACAM1 (NCBI Reference Sequence NP_001703.2; UNIPROT ID P13688) is provided as SEQ ID NO:1 (signal sequence: residues 1-34 of SEQ ID NO:1; Ig-V N domain: residues 35-142 of SEQ ID NO:1). The mature form of CEACAM1 (without signal sequence) is provided as SEQ ID NO:2.

In certain aspects, a human carcinoembryonic-antigen-related cell-adhesion molecule 1 (hCEACAM1) protein, variant or functional fragment thereof, comprises an amino sequence having at least a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the hCEACAM1 protein, variant or functional fragment thereof, comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 1. In certain embodiments, the hCEACAM1 protein, variant or functional fragment thereof, comprises at least a 90%, 91%, 92%, 93%, 94%, 95%, 96%, 97%, 98%, or 99%, sequence identity to SEQ ID NO: 1. In certain embodiments, the hCEACAM1 protein, variant or functional fragment thereof, comprises at least a 95%, 96%, 97%, 98%, or 99%, sequence identity to SEQ ID NO: 1. In certain embodiments, the hCEACAM1 protein, variant or functional fragment thereof, comprises SEQ ID NO: 1.

In another aspect, a hCEACAM1 protein, variant or functional fragment thereof, comprises a 60%, 70%, 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, the hCEACAM1 protein, variant or functional fragment thereof, comprises at least a 75%, 80%, 85%, 86%, 87%, 88%, 89%, 90%, 91%, 92%, 93%, 94%, 95%, 96%, 97%, 98%, or 99%, sequence identity to SEQ ID NO: 2. In certain embodiments, the hCEACAM1 protein, variant or functional fragment thereof, comprises at least a 90%, 91%, 92%, 93%, 94%, 95%, 96%, 97%, 98%, or 99%, sequence identity to SEQ ID NO: 2. In certain embodiments, the hCEACAM1 protein, variant or functional fragment thereof, comprises at least a 95%, 96%, 97%, 98%, or 99%, sequence identity to SEQ ID NO: 2. In certain embodiments, the hCEACAM1 protein, variant or functional fragment thereof, comprises SEQ ID NO: 2.

The major mode of hCEACAM1 binding is homophilic such that it exists on the cell surface as collections of monomers and cis-dimers. This monomer: dimer equilibrium is mainly determined by interactions between the GFCC′ faces of the IgV domains of opposing hCEACAM1 molecules, residues within the transmembrane domain and intracellular calcium concentrations mediated by cellular activation where increased calcium levels promotes hCEACAM1 monomerization. The formation of cell surface hCEACAM1 monomers allows for trans-homophilic or heterophilic interactions critical to the induction of biologic responses. CEACAM1-initiated signaling is triggered by ligation of the N-terminal CEACAM1 IgV domains on opposing cells through high affinity (~450 nM) homophilic mechanisms.

Heterophilic ligands of CEACAM1 include a variety of microbes and host cell proteins such as hCEACAM5, hCEACAM6, CEA (which is not expressed by mouse), the T cell inhibitory and mucin domain containing protein 3 (TIM-3) and programmed cell death protein 1 (PD-1). The tolerance-inducing functions of TIM-3 and CEACAM1 are regulated by CEACAM1 in T cells. Human CEACAM1 is also a well-established microbial receptor and has been demonstrated to bind to multiple pathogenic microorganisms. Pathogenic microbes thus use CEACAM1 to invade the host through binding to epithelial surfaces and subsequently evade host immune responses. The latter occurs through CEACAM1's ability to transduce inhibitory signals via long cytoplasmic domain (CEACAM1-L) containing isoforms that possess ITIMs in immune cells. Once activated, CEACAM1-L interacts with cytoplasmic protein tyrosine kinases and protein tyrosine phosphatases, including SHP-1 and SHP-2, to attenuate cellular activation.

In each case where it has been biochemically or biophysically defined, homophilic and heterophilic ligation depends upon GFCC′ face interactions.

hCEACAM1 has also been shown to form higher-order homo-oligomers and micro-clusters which, when present in a trans-configuration have been hypothesized to be important for strength of signal transduction. In support of hCEACAM1 oligomerization, low resolution (20 Å) molecular tomography studies of liposomal-immobilized rat CEACAM1 (IgV and 3 IgC2 domains), sharing ~43% sequence identify with hCEACAM1 in the IgV domain, revealed multiple states of CEACAM1 that included monomers, dimers, trimers and micro-clusters of closely associated molecules. CEACAM1 higher-order clustering is also supported by studies showing that the propensity for trimer and higher-order oligomer formation significantly increases when proteins with adhesive interfaces are oriented on membrane surfaces.

### CEACAM1 Variants

Provided herein are CEACAM variants. By way of example, hCEACAM1 will be used throughout this disclosure, but is applicable to other CEACAM family members, e.g., CEACAM1, CEACAM3-CEACAM8, CEACAM16 and CEACAM18-21.

In embodiments, the hCEACAM1 variants disclosed herein enhance hCEACAM1's binding affinity and hCEACAM1's ability to induce hCEACAM1-mediated signals, including hCEACAM1-mediated signals on IECs. In embodiments, the hCEACAM1 variants disclosed herein induce the production of AMPs and/or induce hCEACAM1-mediated signals to the immune system to promote inhibition of intestinal inflammation if binding to CEACAM1 molecules located on immune cells.

Provided herein are hCEACAM1 variants or functional fragments thereof that exhibit increased homodimeric binding affinity, increased heterodimeric binding affinity, and/or increased ability to promote CEACAM1-dependent signalling as compared to wild-type hCEACAM1. As used herein, an “hCEACAM1 variant” is a polypeptide that is derived from wild-type hCEACAM1. A “functional fragment” is a fragment of CEACAM1 that retains the desired biological activity, including, but not limited to, the ability of homodimeric, homooligomeric, heterodimeric, or heterooligomeric binding. The hCEACAM1 variant comprises one or more alterations when compared to the parental protein, including, but not limited to amino acid additions, substitutions, insertions, deletions, or posttranslational modifications, wherein the hCEACAM1 variant retains hCEACAM1's biological activity. A substituted or inserted amino acid residue may or may not be one encoded by the genetic code. The hCEACAM1 variant may comprises or consist of a portion of the parental protein that comprises a portion of the parental protein necessary a certain biological activity of the parental protein. For example, the hCEACAM1 variant may comprise or consist of a portion or all of the Ig-V N domain of hCEACAM1. The hCEACAM1 variant may comprise or consist of a portion or all of the extracellular domain of hCEACAM1. In some embodiments, the hCEACAM1 variant comprises one or more conservative mutations as compared to its parental counterpart. In some embodiments, the hCEACAM1 variant comprises 1, 2, 3, 4, 5, 6, 7, 8, 9, or 10 conservative mutations as compared to its parental counterpart (or a relevant portion of the parental counterpart). As used herein, the terms “conservative amino acid substitutions” and “conservative modifications” refer to amino acid modifications that do not significantly affect or alter the function and/or activity of the presently disclosed proteins comprising the amino acid sequence. Such conservative modifications include amino acid substitutions, additions, and deletions. Modifications can be introduced into the proteins of this disclosure by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine.

Provided herein are hCEACAM1 variants or functional fragments thereof that exhibit increased homodimeric binding affinity and/or increased heterodimeric binding affinity as compared to wild-type hCEACAM1 or a relevant portion of wild-type hCEACAM1. As used herein, “binding affinity” may be represented by the equilibrium constant for the equilibrium dissociation (K_{D}) of a first protein to a second protein, is a measure of the binding strength between the two proteins (e.g., two CEACAM1 molecules). K_{D} is calculated as k_{off} (dissociation rate)/kₒₙ (association rate). The smaller the value of the K_{D}, the stronger the binding strength between an antigenic determinant and the antigen-binding molecule. Alternatively, the affinity can also be expressed as the affinity constant (K_{A}), which is 1/K_{D}). In embodiments, the hCEACAM1 variants or functional fragments thereof disclosed herein have a lower dissociation rate k_{off} as compared to wild-type hCEACAM1 or a relevant portion of wild-type hCEACAM1.

In one embodiment, the hCEACAM1 variant or functional fragment thereof is a variant of full length hCEACAM1 (SEQ ID NO:1). In one embodiment, the hCEACAM1 variant or functional fragment thereof is a variant of mature (i.e., with the single peptide cleaved off), but otherwise-length hCEACAM1 (SEQ ID NO:2). In one embodiment, the hCEACAM1 variant or functional fragment thereof is a variant of SEQ ID NO:3 (consisting of the signal peptide (also herein referred to as a signal sequence) and the Ig-V N domain of hCEACAM1). In one embodiment, the hCEACAM1 variant or functional fragment thereof is a variant of SEQ ID NO:4 (consisting of the Ig-V N domain of hCEACAM1). See Table 1 for sequences.

In some embodiments, the hCEACAM1 variant or functional fragment thereof comprises one or more amino acid substitutions and/or chemical modifications at positions R38, V39, D40, G41, Q89 and/or I91. The numbering of the residues is in relation to the residue numbering in the hCEACAM1 sequence without the signal sequence (see SEQ ID NO:2 for the sequence of hCEACAM1 without the signal sequence; see SEQ ID NO:4 for the sequence of hCEACAM1 IgV N-domain without the signal sequence). See residues indicated in bold in SEQ ID NO: 4.

Provided herein is a hCEACAM1 variant or functional fragment thereof that has at least 80%, 85%, 90%, 91%, 92%, 93%, 94%, 95%, 96%, 97%, 98%, or 99% identity to hCEACAM1 variant or functional fragment thereof disclosed herein. Provided herein is hCEACAM1 variant or functional fragment thereof that (1) has at least 80%, 85%, 90%, 91%, 92%, 93%, 94%, 95%, 96%, 97%, 98%, or 99% identity to the relevant portion of hCEACAM1 and (2) comprises one or more amino acid substitutions and/or chemical modifications at positions R38, V39, D40, G41, Q89 and/or I91. Provided herein is hCEACAM1 variant or functional fragment thereof that (1) has at least 80%, 85%, 90%, 91%, 92%, 93%, 94%, 95%, 96%, 97%, 98%, or 99% identity to the relevant portion of hCEACAM1 and (2) comprises one or more amino acid substitutions selected from an R38D substitution, V39C substitution, a D40R substitution, a Q89H substitution, a G41S substitution, a V39F substitution, an I91F substitution, and combinations thereof. Provided herein is hCEACAM1 variant or functional fragment thereof that (1) has at least 80%, 85%, 90%, 91%, 92%, 93%, 94%, 95%, 96%, 97%, 98%, or 99% identity to the relevant portion of hCEACAM1 and (2) comprises one or more amino acid substitutions selected from R38D, V39F, G41S, Q89H, and I91F. As used herein, the term “identity” refers to sequence identity between two nucleic acid molecules or polypeptides. Identity can be determined by comparing a position in each sequence which may be aligned for purposes of comparison. For example, when a position in the compared nucleotide sequence is occupied by the same base, then the molecules are identical at that position. A degree identity between nucleic acid or amino acid sequences is a function of the number of identical or matching nucleotides or amino acids at shared positions. For example, polypeptides having at least 85%, 90%, 95%, 98%, or 99% identity to specific polypeptides described herein and preferably exhibiting substantially the same functions, as well as polynucleotides encoding such polypeptides, are contemplated. Methods and computer programs for determining both sequence identity and similarity are publicly available, including, but not limited to, the GCG program package (Devereux et al., Nucleic Acids Research 12:387, 1984), BLASTP, BLASTN, FASTA (Altschul et al., J. Mol. Biol. 215:403 (1990), and the ALIGN program (version 2.0). The well-known Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

In one embodiment, the hCEACAM1 variant or functional fragment thereof comprises an amino acid substitution and/or chemical modification at position R38. In one embodiment, the hCEACAM1 variant or functional fragment thereof comprises a R38D mutation. In certain embodiments, the hCEACAM1 variant or functional fragment thereof comprises a substitution of R with a polar amino acid amino acid. In certain embodiments, the hCEACAM1 variant or functional fragment thereof comprises a substitution of R with a non-natural amino acid.

In one embodiment, the hCEACAM1 variant or functional fragment thereof comprises an amino acid substitution and/or chemical modification at position V39. In one embodiment, the hCEACAM1 variant or functional fragment thereof comprises a V39A, V39C, or V39F mutation. In certain embodiments, the hCEACAM1 variant or functional fragment thereof comprises a substitution of V39 with alanine, isoleucine, leucine, methionine, phenylalanine, tryptophan or tyrosine.

In one embodiment, the hCEACAM1 variant or functional fragment thereof comprises an amino acid substitution and/or chemical modification at position D40. In one embodiment, the hCEACAM1 variant or functional fragment thereof comprises a D40R mutation. In certain embodiments, the hCEACAM1 variant or functional fragment thereof comprises a substitution of D40 with arginine, asparagine, glutamine, glutamic acid (or glutamate), histidine, lysine, serine, or threonine.

In one embodiment, the hCEACAM1 variant or functional fragment thereof comprises an amino acid substitution and/or chemical modification at position G41. In one embodiment, the hCEACAM1 variant or functional fragment thereof comprises a G41S mutation. In certain embodiments, the hCEACAM1 variant or functional fragment thereof comprises a substitution of G41 with alanine, valine, leucine, isoleucine, proline, phenylalanine, methionine, or tryptophan.

In one embodiment, the hCEACAM1 variant or functional fragment thereof comprises an amino acid substitution and/or chemical modification at position Q89. In one embodiment, the hCEACAM1 variant or functional fragment thereof comprises a Q89H mutation. In certain embodiments, the hCEACAM1 variant or functional fragment thereof comprises a substitution of Q89 with serine, cysteine, threonine, tyrosine, or asparagine.

In one embodiment, the hCEACAM1 variant or functional fragment thereof comprises an amino acid substitution and/or chemical modification at position I91. In one embodiment, the hCEACAM1 variant or functional fragment thereof comprises a I91A or I91F mutation. In certain embodiments, the hCEACAM1 variant or functional fragment thereof comprises a substitution of I91 with glycine, alanine, valine, leucine, proline, phenylalanine, methionine, or tryptophan.

In one embodiment, the hCEACAM1 variant or functional fragment thereof comprises an amino acid substitution and/or chemical modification at position R38 and an amino acid substitution and/or chemical modification at position G51. In certain embodiments, the hCEACAM1 variant or functional fragment thereof comprises a substitution of R with a polar amino acid amino acid and substitution of G41 with alanine, valine, leucine, isoleucine, proline, phenylalanine, methionine, or tryptophan. In certain embodiments, the hCEACAM1 variant or functional fragment thereof comprises a substitution of R with a non-natural amino acid. In one embodiment, the hCEACAM1 variant or functional fragment thereof comprises a R38D and a G41D mutation.

In certain embodiments, the hCEACAM1 variant or functional fragment thereof comprises a substitution of R38, G41, and Q89. In certain embodiments, the hCEACAM1 variant or functional fragment thereof comprises a substitution of R with a polar amino acid amino acid, a substitution of G41 with alanine, valine, leucine, isoleucine, proline, phenylalanine, methionine, or tryptophan, and a substitution of Q89 with serine, cysteine, threonine, tyrosine, or asparagine.

In certain embodiments, the hCEACAM1 variant or functional fragment thereof comprises a substitution of G41 with alanine, valine, leucine, isoleucine, proline, phenylalanine, methionine, or tryptophan, and a substitution of Q89 with serine, cysteine, threonine, tyrosine, or asparagine.

In one embodiment, the hCEACAM1 variant or functional fragment thereof comprises an amino acid substitution at position V39 with alanine, isoleucine, leucine, methionine, phenylalanine, tryptophan or tyrosine and a substitution of I91 with glycine, alanine, valine, leucine, proline, phenylalanine, methionine, or tryptophan.

In one embodiment, the hCEACAM1 variant or functional fragment thereof comprises an amino acid substitution at position V39 with alanine, isoleucine, leucine, methionine, phenylalanine, tryptophan or tyrosine and a substitution of G41 with alanine, valine, leucine, isoleucine, proline, phenylalanine, methionine, or tryptophan.

In one embodiment, the hCEACAM1 variant or functional fragment thereof comprises an amino acid substitution at position V39 with alanine, isoleucine, leucine, methionine, phenylalanine, tryptophan or tyrosine and a substitution of G41 with alanine, valine, leucine, isoleucine, proline, phenylalanine, methionine, or tryptophan and a substitution of Q89 with serine, cysteine, threonine, tyrosine, or asparagine.

Provided herein is hCEACAM1 variant or functional fragment thereof comprising one or more amino acid substitutions selected from Q89H, R38D, R38D/G41S/Q89H, G41S/Q89H, D40R, V39F, I91F, V39F/I91F, V39F/G41S, or V39F/G41S/Q89H.

Provided herein is hCEACAM1 variant or functional fragment thereof comprising one or more amino acid substitutions selected from R38D, V39F, G41S, Q89H, and I91F

TABLE 1
Overview of sequences.

| SEQ | Name for patent | |
|---|---|---|
| ID NO | application | Sequence |
| 1 | CEACAM1 | MGHLSAPLHRVRVPWQGLLLTASLLTFWNPPTTAQLT |
| | (NCBI Reference | TESMPFNVAEGKEVLLLVHNLPQQLFGYSWYKGERVDG |
| | Sequence | NRQIVGYAIGTQQATPGPANSGRETIYPNASLLIQNVTQN |
| | NP_001703.2; | DTGFYTLQVIKSDLVNEEATGQFHVYPELPKPSISSNNSNP |
| | UNIPROT ID | VEDKDAVAFTCEPETQDTTYLWWINNQSLPVSPRLQLSN |
| | P13688) | GNRTLTLLSVTRNDTGPYECEIQNPVSANRSDPVTLNVTY |
| | signal sequence: | GPDTPTISPSDTYYRPGANLSLSCYAASNPPAQYSWLING |
| | residues 1-34 | TFQQSTQELFIPNITVNNSGSYTCHANNSVTGCNRTTVKTI |
| | (bold) | IVTELSPVVAKPQIKASKTTVTGDKDSVNLTCSTNDTGISI |
| | Ig-V N domain: | RWFFKNQSLPSSERMKLSQGNTTLSINPVKREDAGTYWC |
| | residues 35-142 | EVENPISKNQSDPIMLNVNYNALPQENGLSPGAIAGIVIGV |
| | (underlined) | VALVALIAVALACFLHFGKTGRASDQRDLTEHKPSVSNH |
| | | TQDHSNDPPNKMNEVTYSTLNFEAQQPTQPTSASPSLTAT |
| | | EIIYSEVKKQ |
| 2 | mature form of | QLTTESMPFNVAEGKEVLLLVHNLPQQLFGYSWYKGER |
| | CEACAM1 | VDGNRQIVGYAIGTQQATPGPANSGRETIYPNASLLIQNV |
| | (without signal | TQNDTGFYTLQVIKSDLVNEEATGQFHVYPELPKPSISSN |
| | sequence) | NSNPVEDKDAVAFTCEPETQDTTYLWWINNQSLPVSPRL |
| | Ig-V N domain | QLSNGNRTLTLLSVTRNDTGPYECEIQNPVSANRSDPVTL |
| | (underlined) | NVTYGPDTPTISPSDTYYRPGANLSLSCYAASNPPAQYSW |
| | | LINGTFQQSTQELFIPNITVNNSGSYTCHANNSVTGCNRTT |
| | | VKTIIVTELSPVVAKPQIKASKTTVTGDKDSVNLTCSTND |
| | | TGISIRWFFKNQSLPSSERMKLSQGNTTLSINPVKREDAGT |
| | | YWCEVFNPISKNQSDPIMLNVNYNALPQENGLSPGAIAGI |
| | | VIGVVALVALIAVALACFLHFGKTGRASDQRDLTEHKPS |
| | | VSNHTQDHSNDPPNKMNEVTYSTLNFEAQQPTQPTSASP |
| | | SLTATEIIYSEVKKQ |
| 3 | CEACAM1 | MGHLSAPLHRVRVPWQGLLLTASLLTFWNPPTTAQLT |
| | fragment 1 | TESMPFNVAEGKEVLLLVHNLPQQLFGYSWYKGERVDG |
| | Signal sequence | NRQIVGYAIGTQQATPGPANSGRETIYPNASLLIQNVTQN |
| | (bold) | DTGFYTLQVIKSDLVNEEATGQFHVYP |
| | Ig-V N domain | |
| | (underlined) | |
| 4 | CEACAM1 | QLTTESMPFNVAEGKEVLLLVHNLPQQLFGYSWYKGER |
| | fragment 2 | VDGNRQIVGYAIGTQQATPGPANSGRETIYPNASLLIQNV |
| | Ig-V N domain | TQNDTGFYTLQVIKSDLVNEEATGQFHVYP |
| | (underlined) | |
| | Residues V39, | |
| | G41, Q89 and | |
| | I91 (bold and | |
| | italics) | |

In some embodiments, the hCEACAM1 variant or functional fragment thereof is no longer than 140 aa, no longer than 135 aa, no longer than 130 aa, no longer than 125 aa, no longer than 120 aa, no longer than 115 aa, no longer than 110 aa, no longer than 108 aa, no longer than 105 aa, no longer than 100 aa, no longer than 95 aa, no longer than 90 aa, no longer than 85 aa, or no longer than 80 aa. In some embodiments, the hCEACAM1 variant or functional fragment thereof is 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, or 175 aa long.

In some embodiments of the aspects described herein, amino acid sequence modification(s) of the hCEACAM1 variants or functional fragment thereof described herein are contemplated. Amino acid sequence variations for the hCEACAM1 variants or functional fragment thereof can be prepared by introducing appropriate nucleotide changes into the nucleic acid encoding the hCEACAM1 variant or functional fragment thereof, or by peptide synthesis. Such modifications include, for example, deletions from, and/or insertions into and/or substitutions of, residues within the amino acid sequences of the antibody or antigen-binding fragment thereof. Any combination of deletion, insertion, and substitution is made to arrive at the final construct, provided that the final construct possesses the desired characteristics, e.g., binding specificity, inhibition of biological activity, etc. One type of variant is a conservative amino acid substitution variant. These variants have at least one amino acid residue in the hCEACAM1 variant or functional fragment thereof replaced by a different residue that has similar side chain properties. Amino acids can be grouped according to similarities in the properties of their side chains (see Lehninger, BIOCHEMISTRY (2nd ed., Worth Publishers, New York, 1975): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H).

As such, a non-limiting example for a conservative amino acid substitution is one that replaces a non-polar amino acid with another non-polar amino acid.

Alternatively, naturally occurring residues can be divided into groups based on common side-chain properties:
(1) hydrophobic: Ala (A), Val (V), Leu (L), Ile (I), Met (M);
(2) neutral hydrophilic: Ser(S), Thr (T), Cys (C), Asn (N), Gln (Q);
(3) acidic: Asp (D), Glu (E);
(4) basic: Lys (K), Arg (R), His (H);
(5) residues that influence chain orientation: Gly (G), Pro (P);
(6) aromatic: Phe (F), Trp (W), Tyr (Y).

As such, a non-limiting example for a conservative amino acid substitution is one that replaces a hydrophobic amino acid with another hydrophobic amino acid.

Further contemplated are amino acid sequence insertions, which can include amino-and/or carboxyl-terminal fusions ranging in length from one residue to polypeptides containing a hundred or more residues, as well as intrasequence insertions of single or multiple amino acid residues. Modifications of the hCEACAM1 variant or functional fragment thereof disclosed herein include the fusion to the N- or C-terminus of the hCEACAM1 variant or functional fragment thereof to an enzyme or a polypeptide which increases the serum half-life of the hCEACAM1 variant or functional fragment thereof, such as, for example, biotin. Cysteine bond(s) can be added to the hCEACAM1 variant or functional fragment thereof to improve its stability.

In some embodiments, the hCEACAM1 variant or functional fragment thereof is conjugated to an immunoglobulin fragment crystallizable region (Fc region). The Fc region may be derived from an IgG, IgA and IgD antibody. The Fc region may comprise two identical protein fragments, derived from the second and third constant domains of an antibody's two heavy chains. The Fc region may be derived from an IgM and IgE antibody. The Fc region may comprise three heavy chain constant domains (CH domains 2-4) in each polypeptide chain. In an embodiment, one or more amino acid substitutions are introduced into a naturally occurring or synthetic Fc region. For example, one or more cysteine residues can be introduced in the Fc region, thereby allowing interchain disulfide bond formation in this region.

In certain embodiments, the hCEACAM1 variant or functional fragment thereof is conjugated to an Fc receptor.

In some embodiments, it can be desirable to modify the antibodies or antigen-binding fragment thereof that bind to CEACAM1 described herein with respect to effector function, e.g., so as to enhance antigen-dependent cell-mediated cytotoxicity (ADCC) and/or complement dependent cytotoxicity (CDC) of the antibody or antigen-binding fragment thereof. This can be achieved by introducing one or more amino acid substitutions in an Fc region of the antibody or antigen-binding fragment thereof. Alternatively, or additionally, one or more cysteine residues can be introduced in the Fc region, thereby allowing interchain disulfide bond formation in this region. The homodimeric antibody or antigen-binding fragment thereof thus generated can have improved internalization capability and/or increased complement-mediated cell killing and antibody-dependent cellular cytotoxicity (ADCC). See Caron et al., 176 J. Exp. Med. 1191 (1992); Shopes, 148 J. Immunol. 2918 (1992). Homodimeric antibodies with enhanced anti-tumor activity can also be prepared using heterobifunctional cross-linkers as described in Wolff et al., 53 Cancer Res. 2560 (1993). Alternatively, an antibody or antigen-binding fragment thereof can be engineered which has dual Fc regions and can thereby have enhanced complement lysis and ADCC capabilities. See Stevenson et al., 3 Anti-Cancer Drug Design 219 (1989).

In some embodiments, the antibodies or antigen-binding fragments thereof disclosed herein are modified to exhibit effector function reduction or elimination. This can, for example, be accomplished by: (i) reduction or elimination of wild-type mammalian glycosylation of the antibody, (for example, by producing the antibody in an environment where such glycosylation cannot occur, by mutating one or more carbohydrate attachment points such that the antibody cannot be glycosylated, or by chemically or enzymatically removing one or more carbohydrates from the antibody after it has been glycosylated); (ii) by reduction or elimination of the Fc receptor-binding capability of the antibody (for example, by mutation of the Fc region, by deletion within the Fc region or elimination of the Fc region); or (iii) by utilization of an antibody isotype known to have minimal or no effector function (i.e., including but not limited to IgG4). In some embodiments, the heavy chain constant region has one or more of the following mutations: S228P; N297Q; and T299A (numbering according to Kabat). In some embodiments, the heavy chain constant region has one or more of the following mutations: L234A, L235A, and P329G (Kabat EU index numbering).

For example, WO 00/42072 describes antibodies with improved ADCC function in the presence of human effector cells, where the antibodies comprise amino acid substitutions in the Fc region thereof. Preferably, the antibody or antigen-binding fragment thereof with improved ADCC comprises substitutions at positions 298, 333, and/or 334 of the Fc region (Eu numbering of residues). Typically, the altered Fc region is a human IgG1 Fc region comprising or consisting of substitutions at one, two or three of these positions. Such substitutions are optionally combined with substitution(s) which increase Clq binding and/or CDC. Substitutions include an Asn297Ala mutation in IgG1 Fc.

Antibodies with altered Clq binding and/or complement dependent cytotoxicity (CDC) are described in WO 99/51642, U.S. Pat. Nos. 6,194,551, 6,242,195, 6,528,624, and 6,538,124. The antibodies comprise an amino acid substitution at one or more of amino acid positions 270, 322, 326, 327, 329, 313, 333 and/or 334 of the Fc region thereof (Eu numbering of residues).

Antibodies with improved binding to the neonatal Fc receptor (FcRn), and increased half-lives, are described in WO 00/42072 and U.S. Patent Pub. No. 2005/0014934. These antibodies comprise an Fc region with one or more substitutions therein which improve binding of the Fc region to CEACAM1. For example, the Fc region can have substitutions at one or more of positions 238, 250, 256, 265, 272, 286, 303, 305, 307, 311, 312, 314, 317, 340, 356, 360, 362, 376, 378, 380, 382, 413, 424, 428 or 434 (Eu numbering of residues). The preferred Fc region-comprising an antibody variant with improved CEACAM1 binding comprises amino acid substitutions at one, two or three of positions 307, 380 and 434 of the Fc region thereof (Eu numbering of residues). In one embodiment, the antibody or antigen-binding fragment thereof has 307/434 mutations. Engineered antibodies that bind to CEACAM1 with three or more (e.g., four) functional antigen binding sites are also contemplated. See, e.g., U.S. Patent Pub. No. US 2002/0004587.

In certain embodiments, the hCEACAM1 variant or functional fragment thereof is conjugated to one or more agents or moieties. As used herein, the term “agent” or “moiety” is meant to encompass any molecule, chemical entity, composition, drug, therapeutic agent, chemotherapeutic agent, or biological agent capable of preventing, ameliorating, or treating a dysfunction or other medical condition. The term includes small molecule compounds, detectable labels, cytotoxic moieties, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like.

In one embodiment, the hCEACAM1 variant or functional fragment thereof is fused to albumin. The albumin may be fused N-terminally and/or C-terminally to the hCEACAM1 variant or functional fragment thereof.

In some embodiments, the hCEACAM1 variant or functional fragment thereof is conjugated to a functional moiety. Examples of useful functional moieties include, but are not limited to, a blocking moiety, a detectable moiety, a diagnostic moiety, a targeting, and a therapeutic moiety. A functional moiety can have more than one function. Other types of functional moieties are known in the art and can be readily used in the methods and compositions of the present invention based on the teachings contained herein.

Exemplary blocking moieties include moieties of sufficient steric bulk and/or charge such that reduced glycosylation occurs, for example, by blocking the ability of a glycosidase to glycosylate the hCEACAM1 variant or functional fragment thereof. Exemplary blocking moieties include cysteine adducts and PEG moieties. In one embodiment, the blocking moiety is a cysteine, preferably a cysteine that has associated with a free cysteine. Other blocking cysteine adducts include cystine, mixed disulfide adducts, or disulfide linkages. In another embodiment, the blocking moiety is a polyalkylene glycol moiety, for example, a PEG moiety and preferably a PEG-maleimide moiety. Pegylation moieties (or related polymers) can be, for example, polyethylene glycol (“PEG”), polypropylene glycol (“PPG”), polyoxyethylated glycerol (“POG”) and other polyoxyethylated polyols, polyvinyl alcohol (“PVA”) and other polyalkylene oxides, polyoxyethylated sorbitol, or polyoxyethylated glucose. The polymer can be a homopolymer, a random or block copolymer, a terpolymer based on the monomers listed above, straight chain or branched, substituted or unsubstituted as long as it has at least one active sulfone moiety. The polymeric portion can be of any length or molecular weight, but these characteristics can affect the biological properties. Polymer average molecular weights particularly useful for decreasing clearance rates in pharmaceutical applications are in the range of 2,000 to 35,000 Daltons. In addition, if two groups are linked to the polymer, one at each end, the length of the polymer can impact upon the effective distance, and other spatial relationships, between the two groups. Thus, one skilled in the art can vary the length of the polymer to optimize or confer the desired biological activity. PEG is useful in biological applications for several reasons. PEG typically is clear, colorless, odorless, soluble in water, stable to heat, inert to many chemical agents, does not hydrolyze, and is nontoxic. Pegylation can improve pharmacokinetic performance of a molecule by increasing the molecule's apparent molecular weight. The increased apparent molecular weight reduces the rate of clearance from the body following subcutaneous or systemic administration. In many cases, pegylation can decrease antigenicity and immunogenicity. In addition, pegylation can increase the solubility of a biologically-active molecule.

Examples of detectable moieties include fluorescent moieties or labels, imaging agents, radioisotopic moieties, radiopaque moieties, and the like, e.g., detectable labels such as biotin, fluorophores, chromophores, spin resonance probes, or radiolabels. Exemplary fluorophores include fluorescent dyes (e.g., fluorescein, rhodamine, and the like) and other luminescent molecules (e.g., luminal). A fluorophore may be environmentally-sensitive such that its fluorescence changes if it is located close to one or more residues in the modified protein that undergo structural changes upon binding a substrate (e.g., dansyl probes). Exemplary radiolabels include small molecules containing atoms with one or more low sensitivity nuclei (¹³C, ¹⁵N, ²H, ¹²⁵I, ¹²³I, ⁹⁹Tc, ⁴³K, ⁵²Fe, ⁶⁷Ga, ⁶⁸Ga, ¹¹¹In and the like). Other useful moieties are known in the art.

Examples of diagnostic moieties include detectable moieties suitable for revealing the presence of a disease or disorder. Typically, a diagnostic moiety allows for determining the presence, absence, or level of a molecule, for example, a target peptide, protein, or proteins, that is associated with a disease or disorder. Such diagnostics are also suitable for prognosing and/or diagnosing a disease or disorder and its progression.

Examples of therapeutic moieties include, for example, anti-inflammatory agents, anti-cancer agents, anti-neurodegenerative agents, or anti-infective agents. Examples of anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

Cancer therapies in general also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative variant of the foregoing. The combination of chemotherapy with biological therapy is known as biochemotherapy. The chemotherapy may also be administered at low, continuous doses which is known as metronomic chemotherapy.

Yet further combination chemotherapies include, for example, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; carboplatin, procarbazine, plicomycin, gemcitabien, navelbine, farnesyl-protein transferase inhibitors, transplatinum; and pharmaceutically acceptable salts, acids or derivatives of any of the above. In certain embodiments, the compositions provided herein may be used in combination with histone deacetylase inhibitors. In certain embodiments, the compositions provided herein may be used in combination with gefitinib. In other embodiments, the present embodiments may be practiced in combination with Gleevec (e.g., from about 400 to about 800 mg/day of Gleevec may be administered to a patient). In certain embodiments, one or more chemotherapeutic can be used in combination with the compositions provided herein.

In certain embodiments, the hCEACAM1 variant or functional fragment thereof is conjugated to one or more agents comprising immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers Immunomodulatory agents include tumor necrosis factor; interferon alpha, beta, and gamma; IL-2 and other cytokines; F42K and other cytokine analogs; or MIP-1, MIP-1 beta, MCP-1, RANTES, checkpoint inhibitors, and other chemokines.

The term “checkpoint inhibitor” means a group of molecules on the cell surface of CD4⁺ and/or CD8⁺ T cells that fine-tune immune responses by down-modulating or inhibiting an anti-tumor immune response. Immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). “Anti-immune checkpoint inhibitor therapy” refers to the use of agents that inhibit immune checkpoint inhibitors. Inhibition of one or more immune checkpoint inhibitors can block or otherwise neutralize inhibitory signaling to thereby upregulate an immune response in order to more efficaciously treat cancer. Exemplary agents useful for inhibiting immune checkpoint inhibitors include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint inhibitor nucleic acids, or fragments thereof. Exemplary agents for upregulating an immune response include antibodies against one or more immune checkpoint inhibitor proteins block the interaction between the proteins and its natural receptor(s); a non-activating form of one or more immune checkpoint inhibitor proteins (e.g., a dominant negative polypeptide); small molecules or peptides that block the interaction between one or more immune checkpoint inhibitor proteins and its natural receptor(s); fusion proteins (e.g. the extracellular portion of an immune checkpoint inhibition protein fused to the Fe portion of an antibody or immunoglobulin) that bind to its natural receptor(s); nucleic acid molecules that block immune checkpoint inhibitor nucleic acid transcription or translation; and the like. Such agents can directly block the interaction between the one or more immune checkpoint inhibitors and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and anti-CTLA-4 antibodies, either alone or used in combination.

In certain embodiments, the hCEACAM1 variant or functional fragment thereof are conjugated via a linker to the one or more agents.

Single chain peptide linkers, comprised of from one to twenty amino acids joined by peptide bonds, can be used. In certain embodiments, the amino acids are selected from the twenty naturally-occurring amino acids. In certain other embodiments, one or more of the amino acids are selected from glycine, alanine, proline, asparagine, glutamine and lysine. In other embodiments, the linker is a chemical linker. In certain embodiments, said linker is a single chain peptide with an amino acid sequence with a length of at least 25 amino acids, or with a length of 32 to 50 amino acids.

Conjugation may be performed using a variety of chemical linkers. For example, the monovalent binding entity or the fusion protein and the brain effector entity may be conjugated using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). The linker may be a “cleavable linker” facilitating release of the effector entity upon delivery to the brain. For example, an acid-labile linker, peptidase-sensitive linker, photolabile linker, dimethyl linker or disulfide-containing linker (Chari et al, Cancer Res. 52:127-131 (1992); U.S. Pat. No. 5,208,020) may be used.

Covalent conjugation can either be direct or via a linker. In certain embodiments, direct conjugation is by construction of a protein fusion (i.e., by genetic fusion of the two genes encoding the hCEACAM1 variant or functional fragment thereof and the one or more agents expressed as a single protein). In certain embodiments, direct conjugation is by formation of a covalent bond between a reactive group on the hCEACAM1 variant or functional fragment thereof and a corresponding group or acceptor on the agent. In certain embodiments, direct conjugation is by modification (i.e., genetic modification) of one of the two molecules to be conjugated to include a reactive group (as non-limiting examples, a sulfhydryl group or a carboxyl group) that forms a covalent attachment to the other molecule to be conjugated under appropriate conditions. As one non-limiting example, a molecule (i.e., an amino acid) with a desired reactive group (i.e., a cysteine residue) may be introduced. Methods for covalent conjugation of nucleic acids to proteins are also known in the art (i.e., photocrosslinking, see, e.g., Zatsepin et al. Russ. Chem. Rev. 74:77-95 (2005)).

### Nucleic Acids

Also provided herein are nucleic acids encoding the hCEACAM1 variants or functional fragment thereof disclosed herein, as well as vectors, host cells, and expression systems. The term “nucleic acid” as used herein refers to a polymeric form of nucleotides of any length, either ribonucleotides or desoxyribonucleotides. Thus, this term includes, but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases.

The nucleic acids encoding the hCEACAM1 variants or functional fragment thereof disclosed herein may be, e.g., DNA, cDNA, RNA, synthetically produced DNA or RNA, or a recombinantly produced chimeric nucleic acid molecule comprising any of those polynucleotides either alone or in combination. For example, provided is an expression vector comprising a polynucleotide sequence encoding a hCEACAM1 variant or functional fragment thereof disclosed herein described herein operably linked to expression control sequences suitable for expression in a eukaryotic and/or prokaryotic host cell.

The term “vector” refers to a nucleic acid molecule capable of transporting another nucleic acid to which it has been linked. A “vector” includes, but is not limited to, a viral vector, a plasmid, an RNA vector or a linear or circular DNA or RNA molecule which may consists of a chromosomal, non-chromosomal, semi-synthetic or synthetic nucleic acids. In some embodiments, the employed vectors are those capable of autonomous replication (episomal vector) and/or expression of nucleic acids to which they are linked (expression vectors). Large numbers of suitable vectors are known to those of skill in the art and commercially available. Viral vectors include retrovirus, adenovirus, parvovirus (e.g., adeno associated viruses, AAV), coronavirus, negative strand RNA viruses such as orthomyxovirus (e.g., influenza virus), rhabdovirus (e. g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, and spumavirus.

A variety of expression vectors have been developed for the efficient synthesis polypeptides such as hCEACAM1 variants or functional fragment thereof in prokaryotic cells such as bacteria and in eukaryotic systems, including but not limited to yeast and mammalian cell culture systems have been developed. The vectors can comprise segments of chromosomal, non-chromosomal and synthetic DNA sequences. Also provided are cells comprising expression vectors for the expression of the hCEACAM1 variants or functional fragment thereof disclosed herein.

The hCEACAM1 variants or functional fragment thereof are typically produced by recombinant expression. For example, a nucleic acid encoding a hCEACAM1 variant or functional fragment thereof is inserted into an expression vector and operatively linked to an expression control sequence. Expression control sequences include, but are not limited to, promoters (e.g., naturally-associated or heterologous promoters), signal sequences, enhancer elements, and transcription termination sequences. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies.

These expression vectors are typically replicable in the host organisms either as episomes or as an integral part of the host chromosomal DNA. Commonly, expression vectors contain selection markers (e.g., ampicillin-resistance, hygromycin-resistance, tetracycline resistance or neomycin resistance) to permit detection of those cells transformed with the desired DNA sequences (see, e.g., Itakura et al., U.S. Pat. No. 4,704,362).

The expression of the hCEACAM1 variants or functional fragment thereof disclosed herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used.

E. coli is one prokaryotic host particularly useful for cloning the polynucleotides (e.g., DNA sequences) disclosed herein. Other microbial hosts suitable for use include bacilli, such as Bacillus subtilus, and other enterobacteriaceae, such as Salmonella, Serratia, and various Pseudomonas species.

Other microbes, such as yeast, are also useful for expression of the hCEACAM1 variants or functional fragment thereof disclosed herein. Saccharomyces and Pichia are exemplary yeast hosts, with suitable vectors having expression control sequences (e.g., promoters), an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of a hCEACAM1 variant or functional fragment thereof with a specified amino terminus sequence. Moreover, problems associated with retention of initiation codon-derived methionine residues in direct yeast (or bacterial) expression maybe avoided. Sabin et al., 7 Bio/Technol. 705 (1989); Miller et al., 7 Bio/Technol. 698 (1989).

Any of a series of yeast gene expression systems incorporating promoter and termination elements from the actively expressed genes coding for glycolytic enzymes produced in large quantities when yeast is grown in mediums rich in glucose can be utilized to obtain recombinant CEACAM1 antibodies or peptides of the present invention. Known glycolytic genes can also provide very efficient transcriptional control signals. For example, the promoter and terminator signals of the phosphoglycerate kinase gene can be utilized.

Production of hCEACAM1 variants or functional fragment thereof disclosed herein in insects can be achieved. For example, by infecting the insect host with a baculovirus engineered to express a transmembrane polypeptide by methods known to those of skill. See Ausubel et al., 1987, 1993.

In addition to microorganisms, mammalian tissue culture may also be used to express and produce the hCEACAM1 variants or functional fragment thereof disclosed herein. See Winnacker, From Genes to Clones, VCH Publishers, N.Y., N.Y. (1987). Eukaryotic cells can actually be preferred, because a number of suitable host cell lines capable of secreting heterologous proteins have been developed in the art, and include CHO cell lines, various COS cell lines, HeLa cells, 293 cells, myeloma cell lines, transformed B-cells, and hybridomas. Expression vectors for these cells can include expression control sequences, such as an origin of replication, a promoter, and an enhancer (Queen et al., Immunol. Rev. 89:49 (1986)), and necessary processing information sites, such as ribosome binding sites, RNA splice sites, polyadenylation sites, and transcriptional terminator sequences. Preferred expression control sequences are promoters derived from immunoglobulin genes, SV40, adenovirus, bovine papilloma virus, cytomegalovirus and the like. See Co et al., J. Immunol. 148:1149 (1992).

Alternatively, nucleotide sequences encoding hCEACAM1 variants or functional fragment thereof disclosed herein can be incorporated in transgenes for introduction into the genome of a transgenic animal and subsequent expression in the milk of the transgenic animal (see, e.g., Deboer et al., U.S. Pat. No. 5,741,957, Rosen, U.S. Pat. No. 5,304,489, and Meade et al., U.S. Pat. No. 5,849,992). Suitable transgenes include coding sequences for the hCEACAM1 variant or functional fragment thereof in operable linkage with a promoter and enhancer from a mammary gland specific gene, such as casein or beta lactoglobulin.

Additionally, plants have emerged as a convenient, safe and economical alternative main-stream expression systems for polypeptide production, which are based on large scale culture of microbes or animal cells. hCEACAM1 variants or functional fragment thereof disclosed herein can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560 and 6,512,162; and WO 0129242.

The vectors containing the polynucleotide sequences of interest can be transferred into the host cell by well-known methods, which vary depending on the type of cellular host. For example, calcium chloride transfection is commonly utilized for prokaryotic cells, whereas calcium phosphate treatment, electroporation, lipofection, biolistics or viral-based transfection may be used for other cellular hosts. (See generally Sambrook et al., Molecular Cloning: A Laboratory Manual (Cold Spring Harbor Press, 2nd ed., 1989). Other methods used to transform mammalian cells include the use of polybrene, protoplast fusion, liposomes, electroporation, and microinjection (see generally, Sambrook et al., supra). For production of transgenic animals, transgenes can be microinjected into fertilized oocytes, or can be incorporated into the genome of embryonic stem cells, and the nuclei of such cells transferred into enucleated oocytes.

Once expressed, the hCEACAM1 variants or functional fragment thereof disclosed herein can be purified according to standard procedures of the art, including ammonium sulfate precipitation, affinity columns, column chromatography, HPLC purification, gel electrophoresis and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

In certain embodiments, the isolated cell comprises an autologous cell, an allogeneic cell, a haplotype matched cell, a haplotype mismatched cell, a haplo-identical cell, a xenogeneic cell, cell lines or combinations thereof.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be useful where no antibody is available that specifically identifies a cell type in a heterogeneous population, such that separation is carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types. For example, in some aspects, specific subpopulations of T cells, such as cells positive or expressing one or more markers, e.g., CD4⁺CD25⁺, FOXP3⁺ and Helios⁺.

### Methods

Provided herein are methods for enhancing hCEACAM1-mediated induction of anti-inflammatory processes using the hCEACAM1 variants or functional fragments thereof disclosed herein. Provided herein are methods of enhancing immunosuppression using the hCEACAM1 variants or functional fragments thereof disclosed herein. Provided herein are methods of enhancing T cell tolerance using the hCEACAM1 variants or functional fragments thereof disclosed herein.

By “enhancing” is meant the ability to cause an overall increase of as compared to a control that is not treated. In embodiments, the increase is about 2, about 3, about 4, about 5, about 6, about 7, about 8, about 9, about 10, about 15, about 20, about 25, about 30, about 35, about 40, about 45, about 50, about 100, about 200, about 300, about 400, about 500, about 600, about 700, about 800, about 900, about or 1000 fold as compared to a control that is not treated. Methods of measuring the induction of anti-inflammatory processes are well known in the art.

Provided herein are methods for treating autoimmune diseases using the hCEACAM1 variants or functional fragments thereof disclosed herein. Provided herein is a method of treating an autoimmune disease in a subject in need thereof, the method comprising administering to the subject an hCEACAM1 variant or functional fragment thereof disclosed herein. Provided herein is a hCEACAM1 variant or functional fragment thereof for use in treating an autoimmune disease in a subject in need thereof. Provided herein is a hCEACAM1 variant or functional fragment thereof for use in the manufacture of a medicament for treating an autoimmune disease in a subject in need thereof. The hCEACAM1 variant or functional fragment thereof may be conjugated to an Fc region or other agents. By “subject” is meant a mammal, including, but not limited to, a human or non-human mammal, such as a bovine, equine, canine, ovine, or feline, etc. Individuals and patients are also subjects herein. The terms “treat,” “treated,” “treating,” or “treatment” as used herein refer to therapeutic treatment, wherein the object is to slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), or enhancement or improvement of the condition, disorder or disease. Treatment includes eliciting a clinically significant response without excessive levels of side effects. Treatment also includes prolonging survival as compared to expected survival if not receiving treatment. The terms “prevent”, “prevention”, and the like refer to acting prior to overt disease or disorder onset, to prevent the disease or disorder from developing or to minimize the extent of the disease or disorder or slow its course of development.

In embodiments, the methods disclosed herein are useful for treating immune-mediated diseases of the gastrointestinal tract, including, but not limited to, inflammatory bowel disease including Crohn's disease, ulcerative colitis, proctitis and atypical colitis such as lymphocytic colitis, and celiac disease. In embodiments, the methods disclosed herein are useful for treating immune-mediated diseases of the liver including, but not limited to, nonalcoholic steatohepatitis and autoimmune hepatitis. In embodiments, the methods disclosed herein are useful for treating immune-mediated diseases of the stomach including, but not limited to, gastritis. In embodiments, the methods disclosed herein are useful for treating immune-mediated diseases of the pancreas including, but not limited to, acute and chronic pancreatitis and autoimmune pancreatitis. In embodiments, the methods disclosed herein are useful for treating immune-mediated diseases of the esophagus including, but not limited to, esophagitis. In embodiments, the methods disclosed herein are useful for joint diseases including, but not limited to, rheumatoid arthritis. In embodiments, the methods disclosed herein are useful for treating nervous system diseases including, but not limited to, multiple sclerosis and skin conditions. In embodiments, the methods disclosed herein are useful for treating immune-mediated diseases including, but not limited to, dermatitis including, but not limited to, pemphigus and psoriatic disorders.

In other embodiments, the therapeutically effective amount of hCEACAM1 used herein can apply to the prevention and treatment of organ transplant rejection. Such examples include those that involve the liver, lung, heart, kidney or pancreas.

Provided herein are methods for the prevention or treatment of adoptive cell transplant rejection such as that associated with islet cells, stem cells, induced pluripotent stem cells in the treatment of conditions associated with the central nervous system such as Parkinson's disease or heart such as cardiomyopathy.

In other embodiments, are methods for preventing or treating ischemic diseases of the central nervous system such as cerebrovascular accidents, liver or intestines.

In embodiments, a therapeutically effective amount of the hCEACAM1 variant or functional fragment thereof herein is administered to a subject in need thereof. “Therapeutically effective amount” means an amount of the hCEACAM1 variant or functional fragment thereof that, when administered to a subject, is effective in producing the desired therapeutic effect, for example, suppressing inflammation. A therapeutically effective amount as used herein would also include an amount sufficient to delay the development of a symptom of the disease, alter the course of a symptom of disease (for example but not limited to, slow the progression of a symptom of the disease), or reverse a symptom of disease. Thus, it is not possible to specify the exact “therapeutically effective amount” that is effective for all patients and all conditions. However, for any given case, an appropriate “therapeutically effective amount” can be determined by one of ordinary skill in the art using only routine experimentation. Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the agent(s) which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The hCEACAM1 variants or functional fragments thereof described herein can be isolated agents, meaning that the agents are substantially pure and are essentially free of other substances with which they may be found in nature or in vivo systems to an extent practical and appropriate for their intended use. In particular, the agents are sufficiently pure and are sufficiently free from other biological constituents of their host cells so as to be useful in, for example, producing pharmaceutical preparations. Because an isolated agent may be admixed with a pharmaceutically acceptable carrier in a pharmaceutical preparation, the agents may comprise only a small percentage by weight of the preparation.

In some embodiments, the hCEACAM1 variants or functional fragments described herein are administered to a subject by any mode of administration that delivers the agent systemically or to a desired surface or target, and can include, but is not limited to, injection administration and infusion instillation. To the extent that polypeptide agents can be protected from inactivation in the gut, oral administration forms are also contemplated. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the hCEACAM1 variants or functional fragments for use in the methods described herein are administered by intravenous infusion or injection.

The phrases “parenteral administration” and “administered parenterally” as used herein, refer to modes of administration other than enteral and topical administration, usually by injection. The phrases “systemic administration,” “administered systemically,” “peripheral administration” and “administered peripherally” as used herein refer to the administration of an agent other than directly into a target site, tissue, or organ, such that it enters the subject's circulatory system and, thus, is subject to systemic metabolism and other like processes.

In certain embodiments, the hCEACAM1 variant or functional fragment is administered to the subject by intravenous infusion, i.e., introduction of the antibody or antigen-binding fragment thereof into the vein of a mammal over a certain period of time. In certain embodiments, the period of time is about 5 minutes, about 10 minutes, about 30 minutes, about 1 hour, about 2 hours, about 4 hours, or about 8 hours.

In certain embodiments, a dose of a hCEACAM1 variant or functional fragment disclosed herein is administered to a subject every day, every other day, every couple of days, every third day, once a week, twice a week, three times a week, once every two weeks, or once a month. In other embodiments, two, three or four doses of a hCEACAM1 variant or functional fragment disclosed herein is administered to a subject every day, every couple of days, every third day, once a week, once every two weeks or once a month. In some embodiments, a dose(s) of a hCEACAM1 variant or functional fragment disclosed herein is administered for 2 days, 3 days, 5 days, 7 days, 14 days, 21 days or 28 days. In certain embodiments, a dose of a compound or a composition is administered for 1 month, 1.5 months, 2 months, 2.5 months, 3 months, 4 months, 5 months, 6 months or more.

For the clinical use of the methods described herein, administration of a hCEACAM1 variant or functional fragment can include formulation into pharmaceutical compositions or pharmaceutical formulations for parenteral administration, e.g., intravenous or other mode of administration. In some embodiments, a hCEACAM1 variant or functional fragment can be administered along with any pharmaceutically acceptable carrier compound, material, or composition which results in an effective treatment in the subject. Thus, a pharmaceutical formulation for use in the methods described herein can contain an hCEACAM1 variant or functional fragment as described herein in combination with one or more pharmaceutically acceptable ingredients.

Provided herein is a pharmaceutical composition comprising a hCEACAM1 variant or functional fragment disclosed herein and a pharmaceutically acceptable excipient. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase” “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an agent. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) excipients, such as cocoa butter and suppository waxes; (8) oils, such as peanut oil, cottonseed oil, safflower oil, sesame oil, olive oil, corn oil and soybean oil; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (19) pH buffered solutions; (20) polyesters, polycarbonates and/or polyanhydrides; (21) bulking agents, such as polypeptides and amino acids (22) serum components, such as serum albumin, HDL and LDL; (23) C2-C 12 alcohols, such as ethanol; and (24) other non-toxic compatible substances employed in pharmaceutical formulations. Release agents, coating agents, preservatives, and antioxidants can also be present in the formulation. The terms such as “excipient”, “carrier”, “pharmaceutically acceptable carrier” or the like are used interchangeably herein. The agents described herein can be specially formulated for administration of the compound to a subject in liquid form, including those adapted for parenteral administration, for example, by subcutaneous, intramuscular, intravenous or epidural injection as, for example, a sterile solution or suspension, or sustained-release formulation; Additionally, the polypeptide agent can be implanted into a patient or injected using a drug delivery system. See, for example, Urquhart, et al., Ann. Rev. Pharmacol. Toxicol. 24:199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Parenteral dosage forms of an agent can also be administered to a subject by various routes, including, but not limited to, subcutaneous, intravenous (including bolus injection), intramuscular, and intraarterial. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, controlled-release parenteral dosage forms, and emulsions. Suitable vehicles that can be used to provide parenteral dosage forms of the disclosure are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

The pharmaceutical compositions provided herein comprising a hCEACAM1 variant or functional fragment thereof and a pharmaceutically acceptable carrier may comprise the hCEACAM1 variant or functional fragment at various concentrations. For example, the compositions may comprise an hCEACAM1 variant or functional fragment at 10 mg/ml to 200 mg/ml, 25 mg/ml to 130 mg/ml, 50 mg/ml to 125 mg/ml, 75 mg/ml to 110 mg/ml, or 80 mg/ml to 100 mg/ml. The compositions also may comprise an hCEACAM1 variant or functional fragment at about 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, or 150 mg/ml.

In some embodiments, the compositions comprising the hCEACAM1 variant or functional fragment and the pharmaceutically acceptable carrier are lyophilized and provided in a composition for reconstitution prior to administration.

In one aspect, the hCEACAM1 variant or functional fragment thereof is administered with an additional therapeutic agent. Such additional agents include, but are not limited to, a surgical therapy, chemotherapy, radiation therapy, cryotherapy, hyperthermia treatment, phototherapy, radioablation therapy, hormonal therapy, immunotherapy, small molecule therapy, receptor kinase inhibitor therapy, anti-angiogenic therapy, cytokine therapy or biological therapies such as monoclonal antibodies, siRNA, miRNA, antisense oligonucleotides, ribozymes or gene therapy. Without limitation the biological therapy may be a gene therapy, such as tumor suppressor gene therapy, a cell death protein gene therapy, a cell cycle regulator gene therapy, a cytokine gene therapy, a toxin gene therapy, an immunogene therapy, a suicide gene therapy, a prodrug gene therapy, an anti-cellular proliferation gene therapy, an enzyme gene therapy, or an anti-angiogenic factor gene therapy. Immunomodulatory agents include tumor necrosis factor; interferon alpha, beta, and gamma; IL-2 and other cytokines; checkpoint inhibitors; F42K and other cytokine analogs; or MIP-1, MIP-1beta, MCP-1, RANTES, and other chemokines.

The administration of the hCEACAM1 variant or functional fragment thereof and the additional therapeutic agent may be concurrently or consecutively. The administration of the hCEACAM1 variant or functional fragment thereof and the additional therapeutic agent may be separately or as a mixture.

It is to be understood that this invention is not limited to the particular molecules, compositions, methodologies, or protocols described, as these may vary. Any methods and materials similar or equivalent to those described herein can be used in the practice or testing of embodiments of the present invention. It is further to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes those possibilities).

All other referenced patents and applications are incorporated herein by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. This application is filed along with a Sequence Listing File the contents of which are incorporated herein in its entirety.

To facilitate a better understanding of the present invention, the following examples of specific embodiments are given. The following examples should not be read to limit or define the entire scope of the invention.

### EXAMPLES

### Example 1: Structural Analysis of Human CEACAM1 Oligomerization

### Materials and Methods

Crystallization and Structure Refinement of hCEACAM1 Oligomer

Cell pellets containing expressed tagless hCEACAM1 IgV domain was purified using our previously published protocols^{8,10}. Purified protein was concentrated to 15 mg/ml and after the various rounds of the crystallization screening, diffraction quality crystals were grown at 25° C. via sitting drop method in a reservoir solution containing 0.2 M Ammonium sulfate, 0.1 M HEPES pH 7.5, 25% w/v Polyethylene glycol 3,350. The diffraction data was collected at the National Synchrotron Light Source (NSLS, Upton, NY, USA) beamline X25 and processed through iMOSFLM³⁶. The structure of the hCEACAM1 oligomer in the C121 space group was determined using molecular replacement method using polyalanine model of the published hCEACAM1 homodimer crystal structure (PDB code 4QXW) as a search model and refined after various iterative rounds of simultaneous model building to final R and Rfree values of 18.4% and 23.7% using REFMAC³⁷ integrated to CCP4 suite³⁸ and COOT³⁹, respectively. Data collection and refinement statistics are listed in Table 1.

All the figures and comparison of the crystallographic Debye-Waller factor (temperature factor or B factor) of all three molecules of the hCEACAM1 oligomer structure was done using PyMOL (DeLano Scientific). Sequence alignments of hCEACAM family members were done using Clustal Omega⁴⁰. The residues level hydrogen-bond interactions and interface area as observed in the hCEACAM1 oligomer crystal structure for GFCC′ and ABED face was determined using PDB PISA (proteins, interfaces, structures and assemblies) 41.

### Structural Modelling and Attachment of the N-Linked Sugars

Based on the superimposition of mouse CEACAM1 crystal structure (PDB code 1L6Z) onto to all three chains of hCEACAM1 oligomer using Pymol, wherein a conserved N70 glycosylation site residue was resolved in mouse CEACAM1 structure with two molecules of N-linked acetylglucosamine (NAG) NAG and a molecule of β-d-Mannose (BMA), two molecules of NAG and one molecule BMA were modeled on the hCEACAM1 N70 residue first for all three molecules of the hCEACAM1 oligomer. Then based on N70-linked sugar molecules, attachment of the N-linked sugars was performed for N77 and N81 residues of all three molecules of the hCEACAM1 oligomer using Coot. Comparison of the N-linked sugars hCEACAM1 oligomer model and WT homodimer with bound BOG (PDB code 4QXW) was performed using Pymol. In addition to manual modeling, sugar molecule modeling was also performed with the CHARMM glycosylation server³⁰.

UV-Spectroscopy Studies of hCEACAM1 with Various Metal Ions

In this assay, 100 μM concentration of tagless hCEACAM1 purified protein was incubated with various metals (ZnCl₂, NiCl₂, MnCl₂, LiCl₂, CaCl₂),) at a 1:1 or 2.5:1 molar ratio for 30 min. First, OD was performed at 340 nm to measure the aggregation and then after centrifugation for 2 min at 10000 rpm, clear supernatant OD was also performed at 280 nm.

NMR Spectroscopy Studies of hCEACAM1 with Various Metal Ions

NMR samples of 190 μM ¹⁵N-labeled CEACAM1 were prepared with varying concentration of metal ion solutions in 20 mM HEPES, pH 7.4, 150 mM NaCl and 5% D20. The 15N-HSQC experiments were performed at 25° C. on a Bruker Avance II 600 MHz spectrometer equipped with a Prodigy Cryoprobe. The NMR data were acquired with 512 and 80 complex points in the direct HN and indirect 15N dimension respectively. NMR spectra were processed with Bruker Topspin software.

### Statistics and Reproducibility

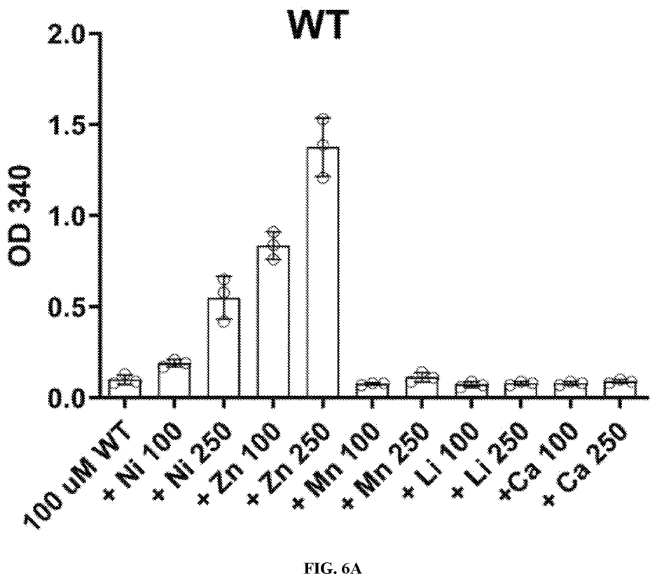

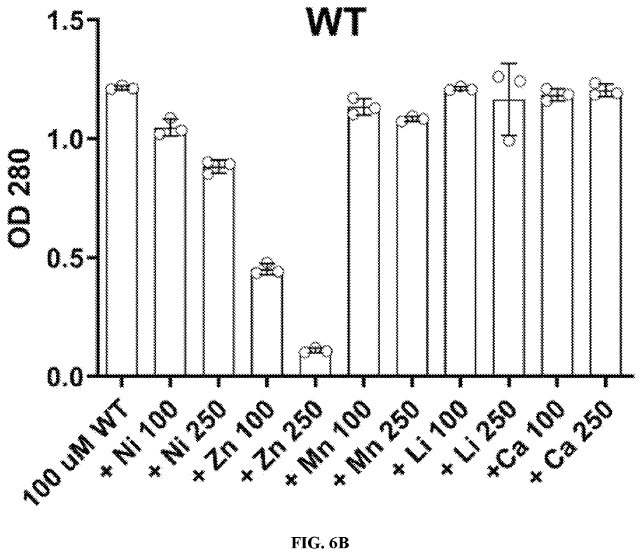

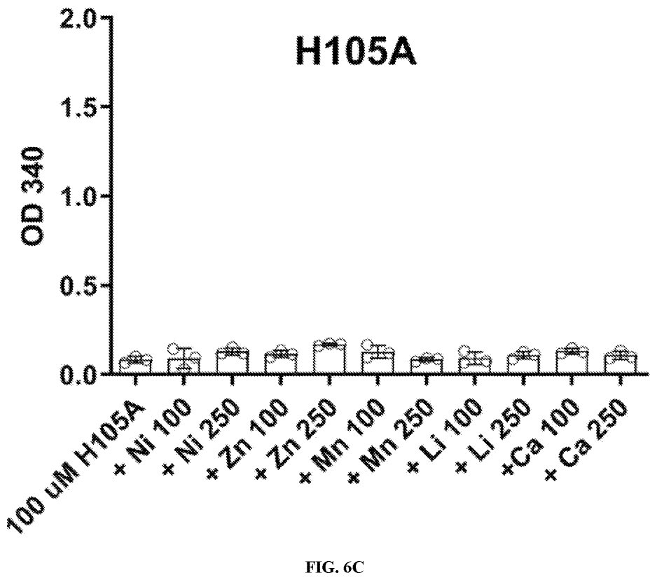

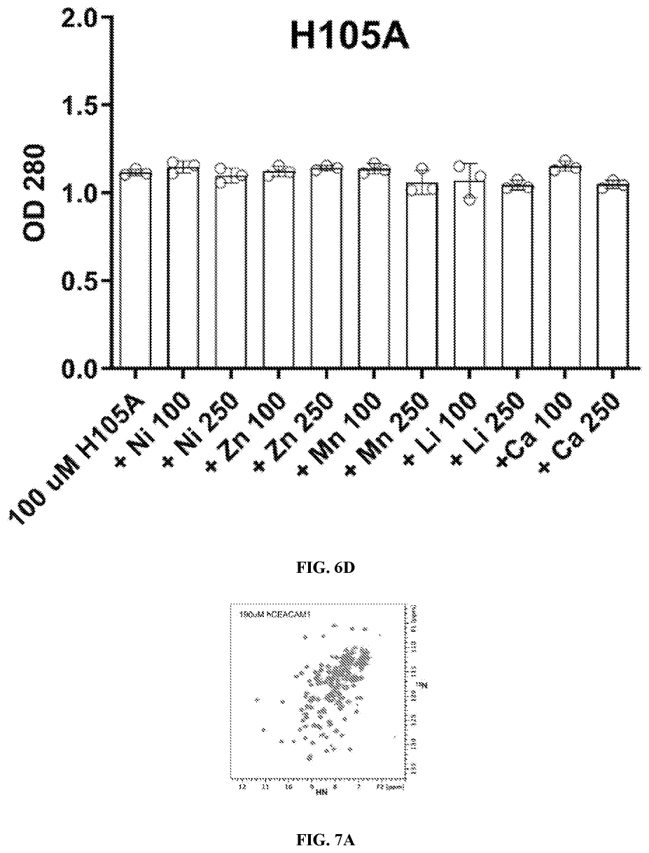

The X-ray data and structure refinement statistics for the hCEACAM1 oligomer crystal structure is shown in Table 1. UV-spectroscopy studies were performed on triplicate samples (n=3) for each metal titration experiment with hCEACAM1 as shown in in FIG. 6. The mean OD values are shown in bar graph with error bars.

### Results

Crystal Structure of hCEACAM1 Oligomer with Two Adhesion Sites and Simultaneous Interactions Through GFCC′ and ABED Face

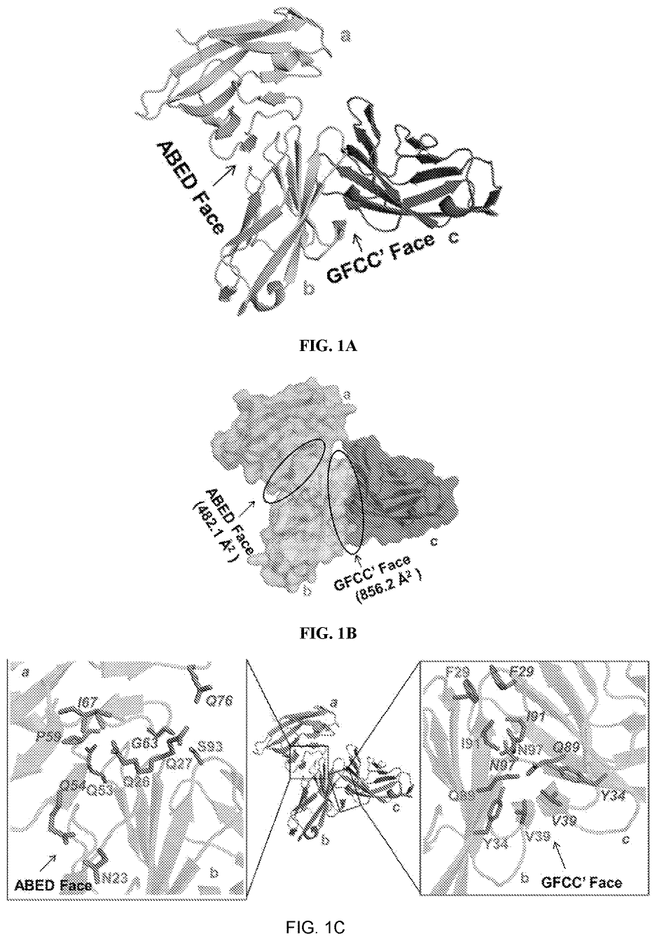

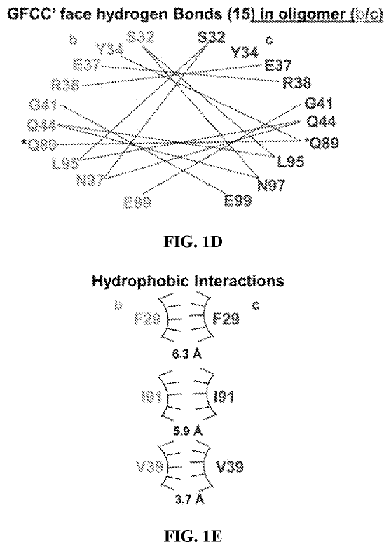

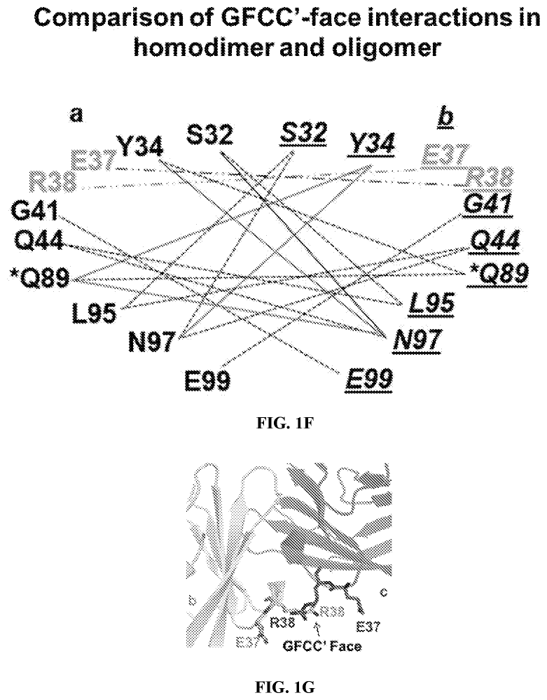

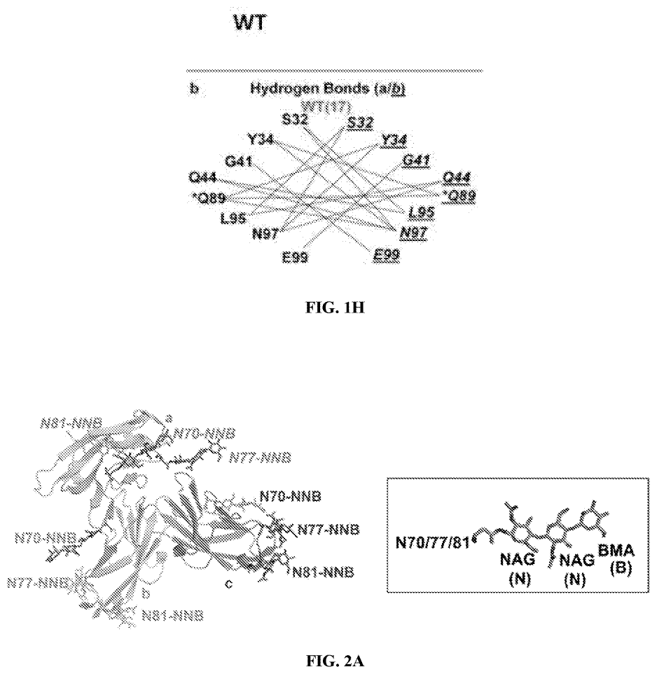

To understand the structural basis for higher-order oligomerization and clustering of hCEACAM1, the crystal structure of a tagless hCEACAM1 IgV domain was solved at 2.2 Å resolution (FIGS. 1A-1C). Crystallographic data collection statistics and structural refinement are summarized in Table 2.

TABLE 2
Data collection and refinement statistics (molecular replacement)
Human CEACAM1 Oligomer (PDB code 7RPP)

| Data Collection | |
|---|---|
| Space group | C121 |
| Cell dimensions | |
| a, b, c (Å) | 73.13, 86.06, 66.08 |
| α, β, γ (°) | 90.0, 100.27, 90.0 |
| Resolution (Å) | 29.86- 2.20 (2.32-2.20) |
| R_{merge} (%) | 13.3 (76.7) |
| I/σl | 7.3 (2.1) |
| Completeness (%) | 100 (100) |
| Redundancy | 4.9 (4.7) |
| Refinement | |
| Resolution (Å) | 29.86- 2.20 (2.32-2.20) |
| No. reflections^{a} | 101329 (13993) |
| R_{work}/R_{free} | 18.4/23.7 |
| No. atoms | |
| Protein | 2520 |
| Ligand/ion | 8 |
| Water | 74 |
| B-factors | |
| Protein | 33.0 |
| Water | 34.73 |
| R.m.s.deviations | |
| Bond-lengths (Å) | 0.016 |
| Bond-angles (°) | 1.78 |

^{a}Values in the parentheses are for highest resolution shell.

The hCEACAM1 oligomer crystal structure revealed three hCEACAM1 molecules (a, b, c) in the asymmetric unit (FIG. 1A) that exhibited an overall similarity in their structure based upon evidence of an anti-parallel beta-sandwich fold of all three molecules compared to a single hCEACAM1 molecule as previously described for a hCEACAM1 homodimer structure (PDB code 4QXW) 10. It was found that molecules (b) and (c) formed a dimer through GFCC′ face interactions, which resembled the WT dimer (PDB code 4QXW) with a C-alpha root mean square deviation (RMSD) of 0.66 Å (over 1489 atoms) and an interaction interface area of 856.2 Å2 (FIG. 1B), that was slightly larger than the interface area of a previously described homodimer (824.6 Å2, PDB code 4QXW) This GFCC′ face interaction of the oligomer was mediated by various hydrogen-bond interactions that included residues Phe (F) 29, S32, Y34, V39, Gly (G) 41, Gln (Q) 44, Gln (Q) 89, Ile (I) 91, N97 and E99 and hydrophobic interactions involving F29, I91 and V39 residues as previously described for a hCEACAM1 GFCC′-mediated homodimer (FIG. 1C).

Changes in the GFCC′ interface were observed in association with an oligomer relative to that involved in a homodimer. Specifically, formation of two new hydrogen-bond interactions was observed between residues E37 and Arg (R) 38 of both molecules (b & c) in the oligomer that were not evident in the crystal structure of a hCEACAM1 dimer (PDB code 4QXW). Specifically, we observed formation of two new hydrogen-bond interactions between residues E37 and Arg(R)38 of both molecules (b & c) in the oligomer that were not evident in the crystal structure of a hCEACAM1 dimer (PDB code 4QXW). In addition, compared to the asymmetrical formation of seven hydrogen-bond interactions of the N97 residue in a hCEACAM1 dimer (PDB code 4QXW), it was observed that the N97 residues of molecule (b) and (c) in the oligomer participated in four symmetrical hydrogen-bond interactions in the formation of the GFCC′ interaction face. This indicates that the GFCC′ face exhibits some degree of flexibility in accommodating the formation of a higher-order oligomer such that it's surface area further increased relative to that observed with a homodimer.

Notably, molecule (b) showed an additional interaction site involving the ABED surface with an interface area of 482.1 Å2 (FIG. 1B) which was mediated by interactions with molecule (a) through residues located at its respective ABED face (FIGS. 1A-1C). This involved six hydrogen-bond interactions between the residues Q26-I67 (using nomenclature convention here and after, where Q26 residue is from molecule (b) and I67 residue in italics is from molecule (a)), Q27-Q76, Q53-Pro(P)59, S93-G63 and N23-Q54 (FIG. 1C). The residues Q26 of molecule (b) and I67 of molecule (a) mediated two hydrogen-bond interactions and formed the central site for the formation of the ABED face, wherein the side-chain atoms NE2 and OE1 of molecule (b) residue Q26 made two hydrogen-bonds of 3.0 Å and 3.1 Å with the main chain oxygen and nitrogen atoms of molecule (a) residue I67, respectively (FIG. 1C). Consistent with the centrality of the Q26-I67 interaction in the formation of an ABED-based interface interactions, these two amino acids were the only contact sites observed in a previously described N97A mutant crystal structure which otherwise disabled hCEACAM1 IgV GFCC′ face-mediated interactions. In addition, molecule (b) residues N23 and Q27 made side-chain to side-chain hydrogen-bond interactions of 2.7 Å and 3.1 Å with molecule (a) residues Q54 and Q76, respectively (FIG. 1C). Side-chain to main-chain backbone hydrogen-bonds of 3 Å between molecule (b) residue Q53 and molecule (a) residue P59 further strengthened the ABED face interactions. In contrast to the GFCC′ interface, the ABED interface did not involve hydrophobic interactions.

Interestingly, molecule (b) residue S93, which is located within the FG loop, made a side-chain to backbone hydrogen-bond interaction of 2.8 Å through molecule (a) residue G63. This unique interaction between a GFCC′ face and ABED face residue demonstrates the coordination between CEACAM1 residues in the formation of oligomers. Taken together, this oligomer crystal structure revealed simultaneous interactions through a primary (GFCC′-GFCC′) and secondary (ABED-ABED) contact at an atomic resolution level thus demonstrating the presence of two adhesion sites on a single hCEACAM1 IgV molecule. Such observations provide a structural foundation for understanding hCEACAM1 oligomerization.

Glycosylation of Residues N70, N77 and N81 Sites are not Predicted to Impede GFCC′ and ABED Face hCEACAM1 Oligomer Interactions

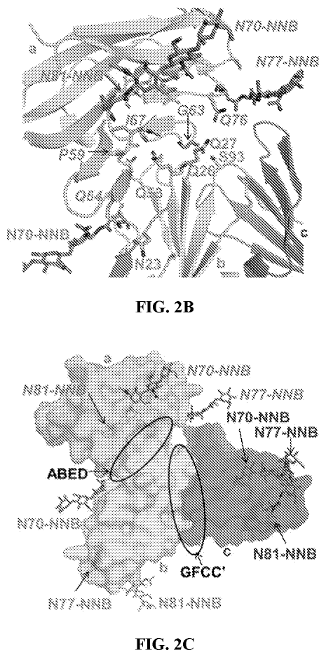

Recent NMR studies have aimed to understand the role of glycosylation in hCEACAM1 GFCC′ face-mediated dimerization with inconclusive results^{27,28} To better understand the potential impact of glycosylation in the formation of hCEACAM1 GFCC′ and ABED face interactions, we modeled the N-linked sugars N-acetylglucosamine (NAG) and β-d-Mannose (BMA) on the N70, N77 and N81 residues within the ABED face residues of all three human CEACAM1 molecules (a, b, c) in the hCEACAM1 oligomeric crystal structure shown above (FIGS. 2A-2C). This manual modeling of sugars was based on the mouse CEACAM1 crystal structure (PDB code 1L6Z) wherein a conserved N70 glycosylation site residue was resolved and shown to be linked to two molecules of NAG and a molecule of BMA29. Consistent with this, the previous crystal structure of a hCEACAM1 dimer (PDB code 4QXW) supported this modeling as it showed binding of octyl beta-D-glucopyranoside (BOG), a glycan mimic, near to residue N70 which overlapped with the N70-linked sugar moieties modeled from mouse CEACAM1¹⁰. A glycosylated model of hCEACAM1 was optimized with N-linked sugars at residues N70, N77 and N81 of each CEACAM1 molecule of the oligomer structure. It was observed that none of the modeled sugar molecules exhibited evidence that they caused steric-hindrance to the ABED face hydrogen-bond interactions (FIGS. 2A-2C). In addition, none of the sugar molecules affected or blocked any of the residues which mediate GFCC′ face interactions in the formation of a homodimer based upon this modeling (FIGS. 2A-2C). Further, as an independent measure of this manual modeling, sugar molecule modeling with the CHARMM glycosylation server³⁰ showed a similar model of hCEACAM1 glycosylation without any steric clashes at the GFCC′ and ABED faces. Consistent with these structural analyses, a previous study showed glycosylation doesn't affect CEACAM1 GFCC′-face mediated interactions with a microbial ligand¹².

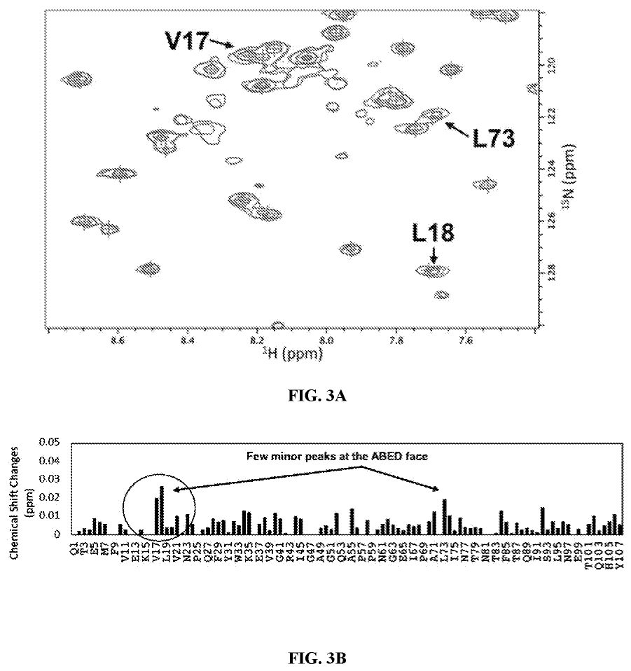

A nuclear magnetic resonance (NMR) spectroscopy study of N15-labeled wild-type hCEACAM1 IgV protein was performed in solution with or without BOG taking advantage of previously described NMR assignments⁸. This showed that addition of BOG caused no peak shift changes at the GFCC′-based dimer interface (FIG. 3A) and only caused a few minor peak shifts that were localized to residues Leu(L)18 and L73 that were not involved in ABED face interactions (FIG. 3B). This was consistent with the observed binding of BOG proximal to residues L18, N70 and L73 in a previously described WT (PDB code 4QXW) or I91A IgV mutant (PDB code 6XNT) crystal structures^{8,10}. Thus, NMR studies of hCEACAM1 and BOG showed no significant changes at the ABED and GFCC′ faces that would anticipate the formation of steric clashing due to glycosylation as predicted by the modeling of sugar molecules onto the hCEACAM1 crystal structure thus allowing for the formation of the oligomeric structure observed.

### Human CEACAM1 GFCC′ Face-Mediated Higher-Order Oligomerization and Micro-Cluster Formation is Supported by Flexible ABED Face Interactions

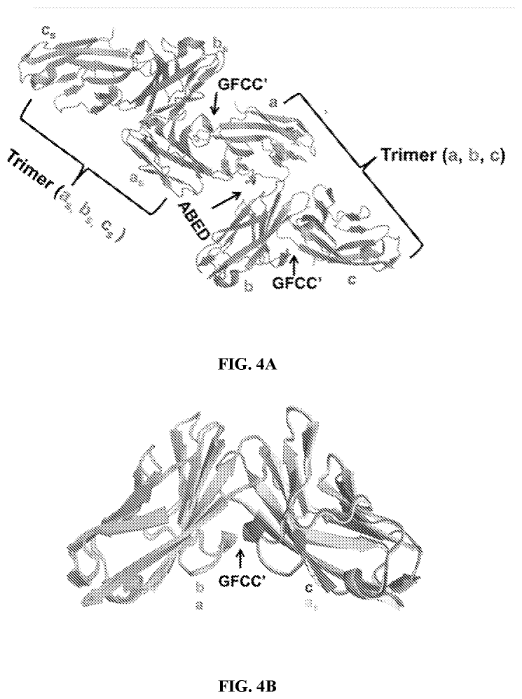

Previous structural studies of wild-type and GFCC′ face mutants of the hCEACAM1 IgV domain and the determination of ligand binding to hCEACAM1 have underscored the importance of the GFCC′ face as representing a central force in hCEACAM1 interactions and monomer:dimer equillibirum^{8,10}. Consistent with the central role of GFCC′ face in dimerization and subsequent oligomerization, GFCC′ face mediated interactions was observed between two oligomers that enabled the formation of higher-order oligomers (FIG. 4A), wherein molecule (a) of the oligomer present in the in the crystal asymmetric unit mediated GFCC′ face interactions with a symmetry mate of molecule a (as) of the symmetry-related second oligomer (FIG. 4A). Superimposition of the oligomer present in the crystal asymmetric unit and symmetry-related second oligomer revealed the GFCC′ face formed in both oligomers were mostly identical (FIG. 4B) with an (RMSD) of 0.6 Å (over 1536 atoms) and with similar GFCC′-face mediated interactions.

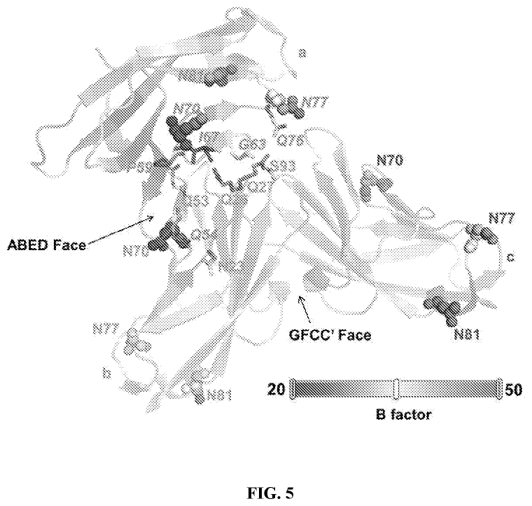

The observation of a secondary interaction site in association with the ABED face as observed in the oligomeric structure provided an opportunity to better understand its role in the formation of higher-order oligomers. The crystallographic Debye-Waller factor (temperature factor or B factor) of all three molecules of the human CEACAM1 crystal structure reported here was therefore determined. This revealed higher thermal motion or dynamic mobility in the side-chains of some of the ABED interface-associated residues (Q27, Q53, Q54) (FIG. 5). In addition, consistent with the notion that sugars exist as flexible molecules, many of the potential sugar attachment site residues (N70, N77 and N81) also exhibited a high B factor (FIG. 5).

Further, it was found that the ABED-associated, hydrogen-bond interactions involve four glutamine residues (Q26, Q53, Q54 and Q76) that form a “four Q-pocket”. The elevated B factors in some of these residues may also contribute to enhanced flexibility of the ABED face through the dual hydrogen-bond donor and acceptor properties of the side-chains associated with these residues. Moreover, as was observed that the four Q-pocket together with the serine (S93) and asparagine (N23) residues which participate in the ABED face interactions are entirely conserved in human CEACAM family member IgV domains, it was anticipated that such flexibility in the formation of oligomers through the ABED face may extend to other hCEACAM family members. This is consistent with the observed structural flexibility revealed by previous molecular tomography studies which showed enough flexibility of the rat CEACAM1 IgV domain to be able to adopt several different conformations. This observed flexibility is anticipated to be important in the formation of higher-order oligomers and micro-clusters where the GFCC′ face serves as dominant initiating force due to its larger interface that includes extensive hydrogen-bond and hydrophobic interactions relative to that associated with the ABED face which is smaller in size with fewer potential interactions and thus serves as an auxiliary, flexible interface.

a Conserved Metal Ion Binding Site in hCEACAM1 and Bridging by Ni⁺⁺ and Zn⁺⁺

One of the important structural features of the hCEACAM1 GFCC′ face is the involvement of the FG loop at the GFCC′-mediated dimeric interface, where residues such as Q89 of the F strand, E99 of the G strand and N97 within the FG loop form a hydrogen-bond network. Of further importance to the structural integrity and conformation of the FG loop and G strand, it was observed that the main-chain nitrogen and carbonyl oxygen atoms of H105 make two hydrogen-bonds with the carbonyl oxygen of F9 and the main chain nitrogen atom of V11, respectively. Moreover, the analysis of hCEACAM1 WT and V39A mutant structures (PDB code 2GK2, 6XNW)^{8,9} and previously described hCEACAM6 WT crystal structures (PDB codes 4WHC, 4Y8A)¹¹ revealed evidence of hexadentate interactions between three H105 and three V106 hCEACAM1 residues contained within the G strands of the FG loop which coordinated with Ni⁺⁺ in the hCEACAM1 structures or Zn⁺⁺ in the hCEACAM6 structure¹¹. This resulted in the bridging of three hCEACAM1 or three hCEACAM6 molecules in the crystal structures, respectively. Interestingly, the ~90% sequence identity observed between the hCEACAM1 and hCEACAM6 IgV domains, which mediates their interactions²², includes conservation of the H105 and V106 residues⁸.

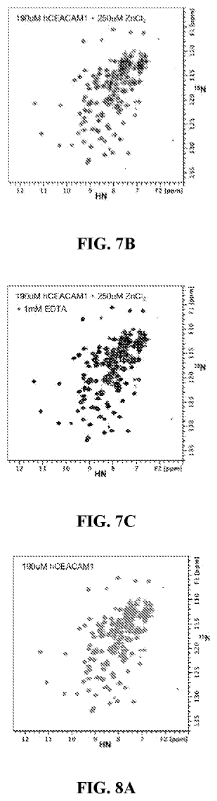

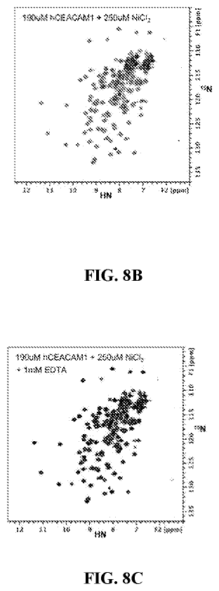

These studies provide evidence that these divalent cations may also contribute to the formation of hCEACAM1 oligomers through interactions in the FG loop. UV-spectroscopy studies of hCEACAM1 with various metal ions was pursued (FIGS. 6A, 6B). In this assay, various metals were titrated at a 1:1 or 2.5:1 molar ratio relative to a 100 μM concentration of hCEACAM1. It was found that Zn⁺⁺ or Ni⁺⁺, but not Ca⁺⁺ or Mn⁺⁺ or Lit ions, showed solution opacity changes and concentration-dependent aggregation of hCEACAM1 with greater induction observed upon addition of Zn⁺⁺ (FIGS. 6A, 6B). To confirm this, NMR spectroscopy studies were carried out. ¹⁵N-labeled WT hCEACAM1 spectra showed loss of hCEACAM1 NMR peak intensities in the presence of varying concentrations of ZnCl₂ without the emergence of new peaks (FIGS. 7A, 7B). Further, it was observed that the NMR samples turned from clear to translucent, to opaque or precipitated with increasing amounts of ZnCl₂ (from 20 μM to 250 μM) with complete reversal through chelation of the divalent cation through addition of 1 mM EDTA (FIG. 7C). Similar solution opacity changes and loss of NMR spectra peaks were also observed for 15N-labeled WT hCEACAM1 in the presence of NiCl₂ (FIGS. 8A-8C). Further, addition of 100 mM imidazole, an analog for histidine side chains, to ¹⁵N-labeled WT hCEACAM1 in the presence of 130 μM NiCl₂ reversed the reduction in the peak intensities of hCEACAM1 observed during binding to the Ni⁺⁺ ions. These structural analyses together with spectroscopy and NMR studies support involvement of Zn⁺⁺ or Ni⁺⁺ binding to histidine residues in the FG loop of the hCEACAM1 IgV domain in the formation of oligomers and micro-clusters.

### DISCUSSION

In this study, the mechanisms involved in the formation of hCEACAM1 multimers were investigated. The atomic level structural studies show that a single hCEACAM1 IgV domain can simultaneously interact with two other hCEACAM1 IgV domains along the dominant GFCC′ and minor ABED face and thus helps to reconcile the presence of ABED (PDB code 2GK2) and GFCC′ (PDB code 4QXW) face-mediated interactions as observed in previously reported crystal structures^{9,10}. This is interesting as previous surface plasmon resonance studies of rat CEACAM1 IgV predicted the existence of two binding sites on the IgV domain consistent with low-resolution topography studies of rat CEACAM1 which show evidence of CEACAM1 trimers and higher order oligomers. The studies herein thus provide confirmation for this and mechanistic insights into this observation based upon the atomic level resolution of a hCEACAM1 oligomer as reported here. Specifically, it was observed that the GFCC′-based interface in this structure exhibited twice the area of the ABED interface. Consistent with this, the GFCC′ interface involved 15 hydrogen-bond and 3 hydrophobic interactions as previously reported for a hCEACAM1 homodimeric IgV domain crystal structure (PDB code 4QXW)¹⁰, compared to 6 hydrogen-bond and no hydrophobic interactions for the ABED interface. The ABED interface in the hCEACAM1 IgV oligomer also included four glutamine residues (four-Q pocket) which were conserved among other hCEACAM family members. This conservation of glutamine residues at this interface is expected to promote flexible hydrogen-bond interactions which would enable clustering of hCEACAM1 and other family members at the cell surface. Subtle differences were also observed in the GFCC′ interface in the context of an oligomer suggesting flexibility in this portion of the molecule that included an increase in the size of its interface area and novel interactions between the R38 and E37 residues that have not been previously observed in a homodimer.

As the structural studies reported here and previously involved a non-glycosylated N-domain that might interfere with multimerization given their location within the IgV domain^{9,27,28} it was also sought to understand the effects of hCEACAM1 glycosylation on the ABED and GFCC′ face interactions. To do so, carbohydrate side-chain modifications were modeled and NMR spectroscopy was performed that probed beta-octylglucoside (BOG) binding to hCEACAM1. This modeling and testing of the assumptions based upon the observed BOG interactions with hCEACAM1 predicted that the presence of sugar molecules at the hCEACAM1 glycosylation sites associated with N70, N77 and N81 would not interfere with the observed ABED and GFCC′ face interactions. Further, B factor analysis of the hCEACAM1 oligomeric structure showed that the sites predicted to be associated with carbohydrate side-chain modifications (N70, N77, N81) as well as the amino acid residues along the ABED interface were characterized by high thermal motion and thus, flexibility. These results are further supported by a recent elegant study published at the time of this manuscript's submission which directly demonstrates by NMR and structural (PDB code 7MU8) observations that dimer formation through hCEACAM1 GFCC′ face interactions is observed in the presence of partially glycosylated hCEACAM1³¹.

Finally, the role of metals on hCEACAM1 multimerization was examined as previous studies showed Ni⁺⁺ and Zn⁺⁺-mediated bridging of 3 hCEACAM1 molecules by coordination through histidine and valine residues in the G strands of the FG loops^{8,9}. The presence of Zn⁺⁺ coordination of the same G strand residues in a previously reported structure of hCEACAM611 was noticed. As such, the biochemical role of Zn⁺⁺ and Ni⁺⁺ in regulating hCEACAM1 multimerization was also investigated using UV spectroscopy and NMR studies. This revealed evidence of reversible Zn⁺⁺- and Ni⁺⁺-mediated oligomerization of hCEACAM1 based upon competition with EDTA or imidazole, a histidine residue mimetic. The hCEACAM1 cytoplasmic tail has been shown to bind calmodulin in a Ca⁺⁺-dependent manner which downregulates cis homodimerization of hCEACAM132. This provides evidence that oligomerization facilitated by these metals is a concentration dependent, dynamic process and further that other FG-loop associated factors regulate hCEACAM1 multimer formation involving the IgV domain. Similar Zn⁺⁺ and Ni⁺⁺-mediated dynamic and reversible oligomerization in coordination through histidine residues was observed with APOBEC3G (A3G), a DNA cytidine deaminase and with carbon monoxide dehydrogenase (CooJ)^{34,35}, respectively, using similar experimental approaches as reported here. In these studies, NMR titration experiments showed 50 mM Zn⁺⁺-dependent A3G (300 μM) oligomerization and transmission electron microscopy (TEM) showed 1 mM Ni⁺⁺-dependent CooJ (100 μM) oligomerization, which were reversible in both cases in the presence of EDTA 33-35.

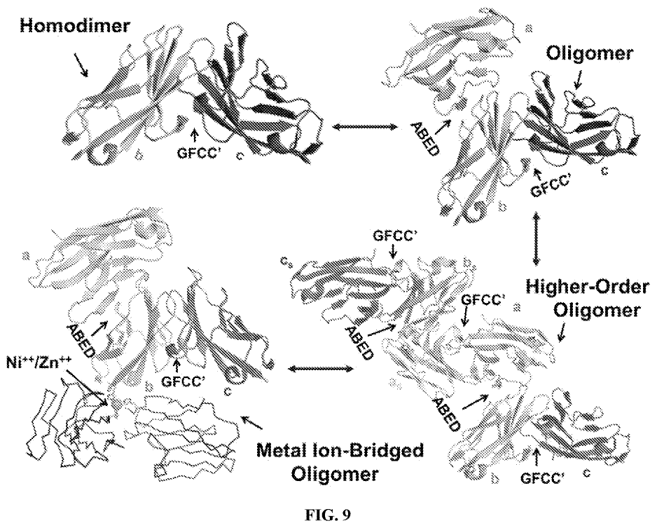

Overall, these studies provide structural insights into how hCEACAM1 could exist as heterogeneous mixtures of oligomers and in diverse conformational states on the cell surface through primary GFCC′ and secondary ABED interfaces that are mediated by the IgV domain which was shown to be able to simultaneously bind two hCEACAM1 molecules (FIG. 9). In this model, homodimerization through the GFCC′ face enables highly flexible ABED-mediated interactions to form oligomers and higher-order oligomers. These are formed by symmetry mates of molecules a, b and c as observed in the crystal structure and are also based upon GFCC′ face interactions which may be further facilitated by metal ions such as Zn⁺⁺ and Ni⁺⁺ (FIG. 9). It is not known how this model relates to cis or trans interactions or their functional implications, however. It is equally plausible that it represents a means to direct hCEACAM1 to an inactive state on the cell surface when in cis or to intracellular signal transduction when the GFCC′ and consequently ABED interactions occur in trans across two cells. Nonetheless, the proposed model and the results of the biophysical and structural studies herein, help to better understand previous observations and provide new insights into the structural basis for the formation of hCEACAM1 oligomers with implications for other hCEACAM family members. They may also potentially provide insights into the nature of the lower affinity heterophilic ligands for hCEACAM1 such as PD1¹⁴ and TIM-3¹⁰ which are likely to be highly influenced by the effects of avidity that would be facilitated by hCEACAM1 oligomerization.

### Example 2: Generation of High Affinity Dimer CEACAM1 Variants

Utilizing the inventor's atomic level understanding of the CEACAM1-GFCC′ face by resolving crystal structures and NMR-assignments of hCEACAM1 Ig-V domain and various mutants (PDB ID 4QXW (hCEACAM1 wild-type (WT)), PDB ID 6XNO (hCEACAM1 E99A), PDB ID 6XNT (hCEACAM1 I91A), PDB ID 6XNW (hCEACAM1 V39A), PDB ID 6X01 (CEACAM1 N97A), BMRB ID 50368 (hCEACAM1 IgV WT dimer), BMRB ID 50366 (hCEACAM1 N97A), and PDB ID 7RPP (hCEACAM1 oligomer)), hCEACAM1 variants with increased binding affinity were engineered.

Provided herein are two classes of hCEACAM1 variants. One class of hCEACAM1 variants shows increased affinity in the heterodimer form (e.g. a soluble hCEACAM1 variant is administered to a patient and binds to a hCEACAM1 interaction partner located on a patient's cells). hCEACAM1 amino acid modifications in this class include modifications of R38, D40, G41, and Q89. The other class of hCEACAM1 variants shows increased affinity in the homodimer form (e.g. a soluble hCEACAM1 variant is administered to a patient and binds to a hCEACAM1 molecule located on a patient's cells). hCEACAM1 amino acid modifications in this class include modifications of V39 and I91.

### Class 1 Modifications

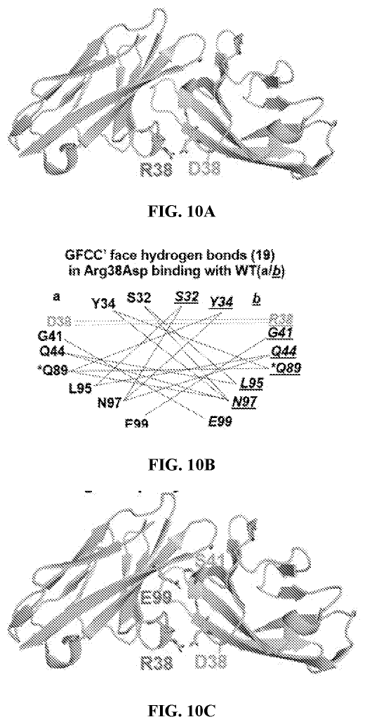

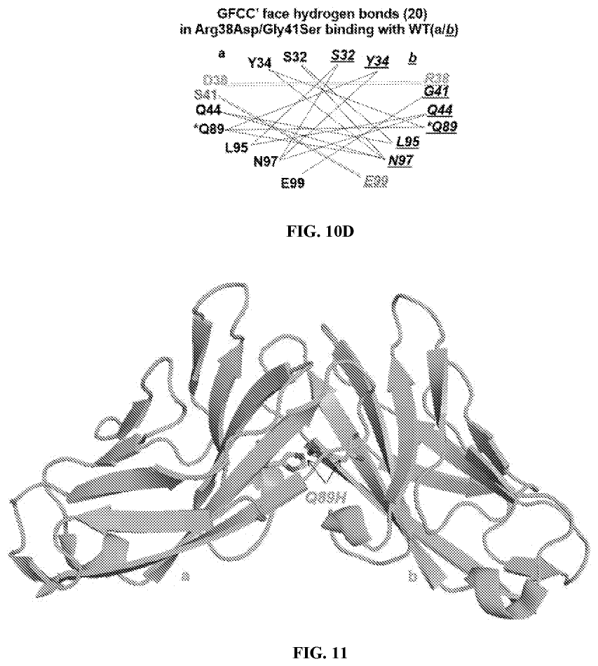

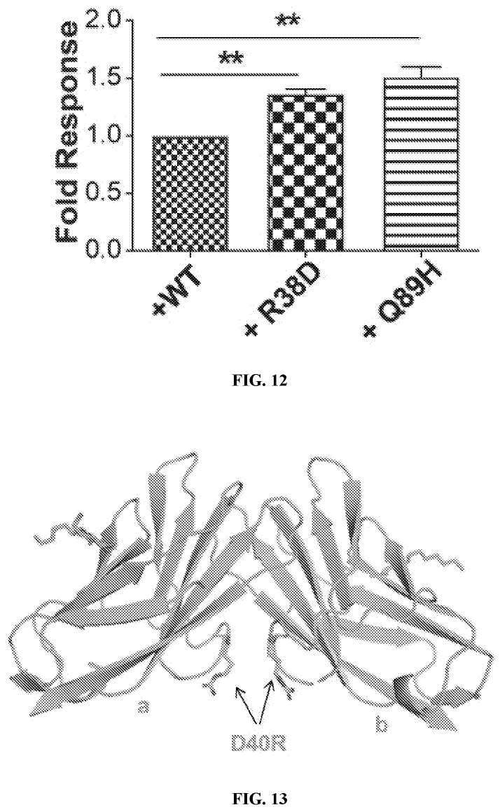

As discussed in more detail in Example 1, the resolution of a hCEACAM1 oligomer crystal structure (PDB ID 7RPP) shows for the presence of not only one, but two binding interfaces on hCEACAM1. The dominant face is GFCC′ face and contains the hCEACAM1 common ligand binding site for hCEACAM1, hTIM-3 and microbes (including Helicobacter pylori HopQ protein and group B Streptococcus Ig-like protein). The second minor face is ABED face. In solving the hCEACAM1 oligomer structure, it was observed that residue R38 mediated two hydrogen-bonded interactions with residue E37. Of note, it was found that these residues did not mediate any hydrogen-bonded interactions in homodimer structure (PDB ID 4QXW) although located at the center of the GFCC′ face. This R38:E37 interaction in an oligomer but not in homodimer structure demonstrates flexibility in the GFCC′ face interactions. As such, class 1 hCEACAM1 variant R38D was engineered. This mutation introduces a salt-bridge in the GFCC′ face if binding to a wild-type (WT) hCEACAM1 protein. The crystal structure was this mutant was resolved at ~3.3 Å. FIG. 10A shows a heterodimer comprising a wildtype hCEACAM1 monomer and a R38D monomer. The R38D substitution increased hydrogen bonding and additional salt-bridge interactions at the GFCC′ face. Specifically, the number of hydrogen bonds between the two hCEACAM1 monomers in the hCEACAM1 dimer was increased from 17 in the WT:WT configuration to 19 in the WT:R38D configuration. The number of salt bridges between the two hCEACAM1 monomers in the hCEACAM1 dimer was increased from 0 in the WT:WT configuration to 5 in the WT:R38D configuration (see also FIG. 10B). The stability of macromolecular complex in a biological system is governed by the following physico-chemical properties: (1) free energy of formation, (2) solvation energy gain, (3) interface area. (4) hydrogen bonds, (5) salt bridges across the interface, and (6) hydrophobics pecificity. As such, the stability of dimer complexes can be assessed using the crystal structure for the dimer and the program PDB-PISA. PDB-PISA analysis of the hCEACAM1 wildtype and hCEACAM1 R38D revealed an increase in the PDB dimer formation score from 0.89 (WT) to 0.91 (R38D). ELISA binding assays confirmed an increased binding affinity between WT:R38D as compared to WT:WT (FIG. 12).

The R38D hCEACAM1 variant was further combined with a G41S mutation, which introduces additional hydrogen-bonded interactions between the S41 residue in hCEACAM1 G41S and the E99 side chain in WT hCEACAM1. FIG. 10C shows a heterodimer comprising a wildtype hCEACAM1 monomer and a R38D G41S monomer. The introduction of the additional amino acid substitution added an additional hydrogen bond (compare 17 hydrogen bonds in the WT:WT configuration and 20 in the WT:R38D/G41S configuration (FIG. 10D).

The crystal structure of another class 1 variant, hCEACAM1 Q89H, was resolved at a resolution of 1.7 Å (FIG. 11). Residue Q89 is also contained within the GFCC′ face and a Q89H substitution enhanced the hydrogen-bonded interactions at the homodimer interface due to the dual hydrogen-bond donor/acceptor profile of H89 residue side chain nitrogen atoms in the Q89H variant. Further, PDB-PISA analysis of the hCEACAM1 wildtype and hCEACAM1 Q89H revealed an increase in the PDB dimer formation score from 0.89 (WT) to 0.96 (Q89H). ELISA binding assays confirmed an increased binding affinity between WT:Q89H as compared to WT:WT (FIG. 12).

The crystal structure of hCEACAM1 variant D40R was resolved at a resolution of 1.7 Å (FIG. 13). PDB-PISA analysis of the hCEACAM1 wildtype and hCEACAM1 D40R revealed an increase in the PDB dimer formation score from 0.89 (WT) to 0.93 (D40R).

### Class 2 Modifications

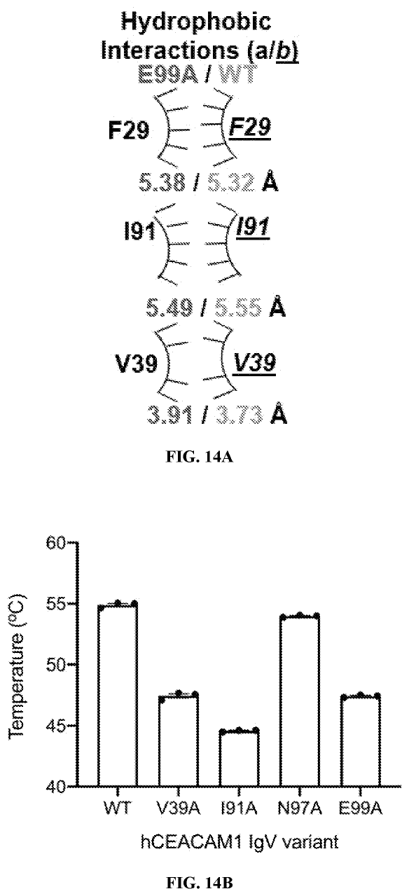

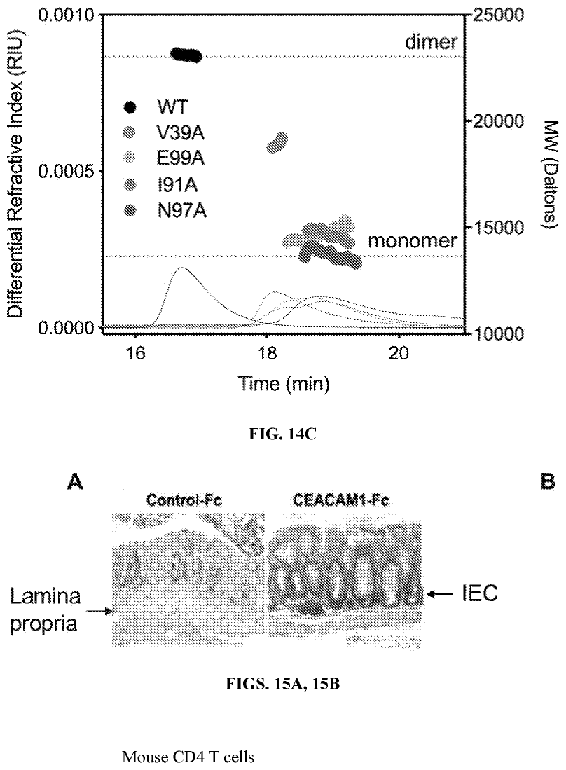

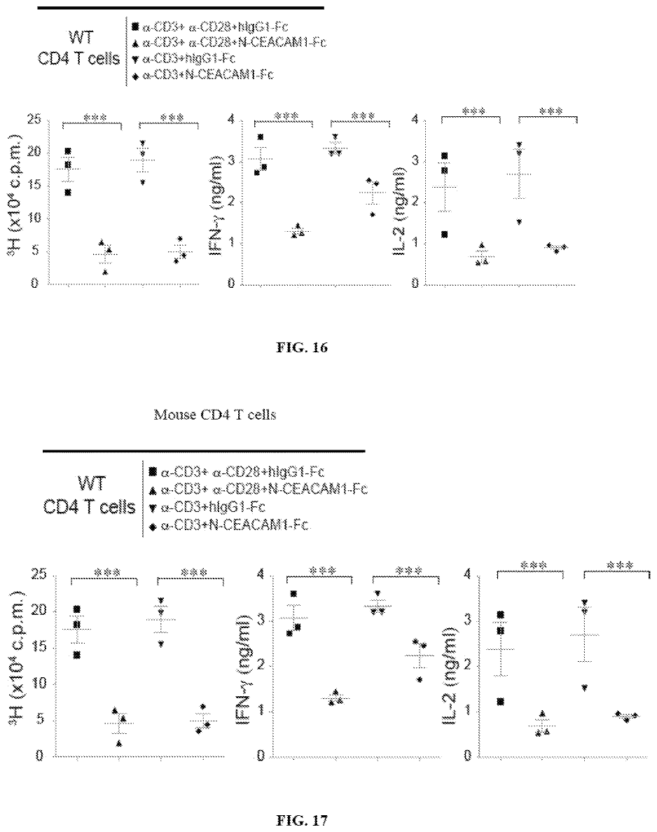

Residues V39 and I91 play a central role in mediating hydrophobic interactions at human CEACAM1 dimerization interface and based on the dimer crystal structure (PDB ID 4QXW). The presence of phenylalanine at position F29, valine at position 39, and isoleucine at position 91 provides hydrophobic force at dimer interface (FIG. 14A). Consistent with this, mutations of V39 or I91 to alanine (a smaller hydrophobic amino acid) weaken the hCEACAM1 homodimer (FIGS. 14B and 14C). Conversely, substitution of residues V39 and I91 to phenylalanine further increase dimerization affinity due to higher hydrophobic nature of phenylalanine and confirmed by structure analysis (V39F; I91F).

While the foregoing written description of the invention enables one of ordinary skill to make and use what is considered presently to be the best mode thereof, those of ordinary skill will understand and appreciate the existence of variations, combinations, and equivalents of the specific embodiments, methods, and examples herein.

### REFERENCES

1. Gray-Owen, S. D. & Blumberg, R. S. CEACAM1: contact-dependent control of immunity. Nat Rev Immunol 6, 433-446 (2006).
2. Kim, W. M., Huang, Y. H., Gandhi, A. & Blumberg, R. S. CEACAM1 structure and function in immunity and its therapeutic implications. Seminars in Immunology (2019) doi: 10.1016/j.smim.2019.101296.
3. Kammerer, R. & Zimmermann, W. Coevolution of activating and inhibitory receptors within mammalian carcinoembryonic antigen families. BMC Biology (2010) doi: 10.1186/1741-7007 August 12.
4. Huang, J., Hardy, J. D., Sun, Y. & Shively, J. E. Essential role of biliary glycoprotein (CD66a) in morphogenesis of the human mammary epithelial cell line MCF10F. Journal of Cell Science (1999).
5. Ergün, S. et al. CEA-related cell adhesion molecule 1: A potent angiogenic factor and a major effector of vascular endothelial growth factor. Molecular Cell (2000) doi: 10.1016/S1097-2765(00)80426-8.
6. Beauchemin, N. et al. Redefined nomenclature for members of the carcinoembryonic antigen family. Experimental Cell Research (1999) doi: 10.1006/excr.1999.4610.
7. Beauchemin, N. et al. Redefined nomenclature for members of the carcinoembryonic antigen family. Exp Cell Res 252, 243-249 (1999).
8. Gandhi, A. K. et al. Structural basis of the dynamic human CEACAM1 monomer-dimer equilibrium. Communications Biology 4, (2021).
9. Fedarovich, A., Tomberg, J., Nicholas, R. A. & Davies, C. Structure of the N-terminal domain of human CEACAM1: binding target of the opacity proteins during invasion of Neisseria meningitidis and N. gonorrhoeae. Acta Crystallogr D Biol Crystallogr 62, 971-979 (2006).
10. Huang, Y. H. et al. CEACAM1 regulates TIM-3-mediated tolerance and exhaustion. Nature 517, 386-390 (2015).
11. Bonsor, D. A., Günther, S., Beadenkopf, R., Beckett, D. & Sundberg, E. J. Diverse oligomeric states of CEACAM IgV domains. Proceedings of the National Academy of Sciences of the United States of America (2015) doi: 10.1073/pnas.1509511112.
12. Bonsor, D. A. et al. The Helicobacter pylori adhesin protein HopQ exploits the dimer interface of human CEACAMs to facilitate translocation of the oncoprotein CagA. The EMBO Journal (2018) doi: 10.15252/embj.201798664.
13. Gandhi, A. K. et al. High resolution X-ray and NMR structural study of human T-cell immunoglobulin and mucin domain containing protein-3. Scientific Reports (2018) doi: 10.1038/s41598-018-35754-0.
14. Wojtowicz, W. M. et al. A Human IgSF Cell-Surface Interactome Reveals a Complex Network of Protein-Protein Interactions. Cell 182, (2020).
15. Korotkova, N. et al. Binding of Dr adhesins of Escherichia coli to carcinoembryonic antigen triggers receptor dissociation. Mol Microbiol 67, 420-434 (2008).
16. Königer, V. et al. Helicobacter pylori exploits human CEACAMs via HopQ for adherence and translocation of CagA. Nature Microbiology (2016) doi: 10.1038/nmicrobiol.2016.188.
17. Brewer, M. L. et al. Fusobacterium spp. target human CEACAM1 via the trimeric autotransporter adhesin CbpF. Journal of Oral Microbiology (2019) doi: 10.1080/20002297.2018.1565043.
18. Klaile, E. et al. Binding of Candida albicans to human CEACAM1 and CEACAM6 modulates the inflammatory response of intestinal epithelial cells. mBio (2017) doi: 10.1128/mBio.02142-16.
19. Watt, S. M. et al. Homophilic adhesion of human CEACAM1 involves N-terminal domain interactions: structural analysis of the binding site. Blood 98, 1469-1479 (2001).
20. Zhang, D. et al. Identification and characterization of M6903, an antagonistic anti-TIM-3 monoclonal antibody. OncoImmunology 9, (2020).
21. Sabatos-Peyton, C. A. et al. Blockade of Tim-3 binding to phosphatidylserine and CEACAM1 is a shared feature of anti-Tim-3 antibodies that have functional efficacy. OncoImmunology 7, (2018).
22. Pinkert, J. et al. T cell-mediated elimination of cancer cells by blocking CEACAM6-CEACAM1 interaction. OncoImmunology 11, (2022).
23. Muller, M. M., Klaile, E., Vorontsova, O., Singer, B. B. & Obrink, B. Homophilic adhesion and CEACAM1-S regulate dimerization of CEACAM1-L and recruitment of SHP-2 and c-Src. J Cell Biol 187, 569-581 (2009).
24. Klaile, E. et al. The CEACAM1 N-terminal Ig domain mediates cis- and trans-binding and is essential for allosteric rearrangements of CEACAM1 microclusters. Journal of Cell Biology (2009) doi: 10.1083/jcb.200904149.
25. Patel, P. C. et al. Inside-out signaling promotes dynamic changes in the carcinoembryonic antigen-related cellular adhesion molecule 1 (CEACAM1) oligomeric state to control its cell adhesion properties. J Biol Chem 288, 29654-29669 (2013).
26. Grasberger, B., Minton, A. P., DeLisi, C. & Metzger, H. Interaction between proteins localized in membranes. Proceedings of the National Academy of Sciences of the United States of America 83, (1986).
27. Zhuo, Y., Yang, J. Y., Moremen, K. W. & Prestegard, J. H. Glycosylation alters dimerization properties of a cell-surface signaling protein, carcinoembryonic antigen-related cell Adhesion Molecule 1 (CEACAM1). Journal of Biological Chemistry (2016) doi: 10.1074/jbc.M116.740050.
28. Zhuo, Y., Yang, J.-Y., Moremen, K. W. & Prestegard, J. H. Correction: Glycosylation alters dimerization properties of a cell-surface signaling protein, carcinoembryonic antigen-related cell adhesion molecule 1 (CEACAM1). Journal of Biological Chemistry (2020) doi: 10.1074/jbc.aac120.013039.
29. Tan, K. et al. Crystal structure of murine sCEACAM1a [1,4]: a coronavirus receptor in the CEA family. Embo J 21, 2076-2086 (2002).
30. Jo, S., Kim, T., Iyer, V. G. & Im, W. CHARMM-GUI: A web-based graphical user interface for CHARMM. Journal of Computational Chemistry 29, (2008).
31. Belcher Dufrisne, M. et al. Human CEACAM1 N-domain dimerization is independent from glycan modifications. Structure (2022) doi: 10.1016/j.str.2022.02.003.
32. Edlund, M., Blikstad, I. & Obrink, B. Calmodulin binds to specific sequences in the cytoplasmic domain of C-CAM and down-regulates C-CAM self-association. J Biol Chem 271, 1393-1399 (1996).
33. Shandilya, S. M. D. et al. Crystal Structure of the APOBEC3G Catalytic Domain Reveals Potential Oligomerization Interfaces. Structure 18, (2010).
34. Alfano, M., Pérard, J. & Cavazza, C. Nickel-induced oligomerization of the histidine-rich metallochaperone CooJ from Rhodospirillum rubrum. Inorganics 7, (2019).
35. Alfano, M. et al. The carbon monoxide dehydrogenase accessory protein CooJ is a histidine-rich multidomain dimer containing an unexpected Ni(II)-binding site. Journal of Biological Chemistry 294, (2019).
36. Battye, T. G. G., Kontogiannis, L., Johnson, O., Powell, H. R. & Leslie, A. G. W. iMOSFLM: A new graphical interface for diffraction-image processing with MOSFLM. Acta Crystallographica Section D: Biological Crystallography (2011) doi: 10.1107/S0907444910048675.
37. Murshudov, G. N., Vagin, A. A. & Dodson, E. J. Refinement of macromolecular structures by the maximum-likelihood method. Acta Crystallographica Section D: Biological Crystallography (1997) doi: 10.1107/S0907444996012255.
38. Winn, M. D. et al. Overview of the CCP4 suite and current developments. Acta Crystallographica Section D: Biological Crystallography (2011) doi: 10.1107/S0907444910045749.
39. Emsley, P., Lohkamp, B., Scott, W. G. & Cowtan, K. Features and development of Coot. Acta Crystallographica Section D: Biological Crystallography (2010) doi: 10.1107/S0907444910007493.
40. Sievers, F. et al. Fast, scalable generation of high-quality protein multiple sequence alignments using Clustal Omega. Molecular Systems Biology (2011) doi: 10.1038/msb.2011.75.1
41. Krissinel, E. & Henrick, K. Inference of Macromolecular Assemblies from Crystalline State. Journal of Molecular Biology (2007) doi: 10.1016/j.jmb.2007.05.022.

### OTHER EMBODIMENTS

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference. By their citation of various references in this document, Applicants do not admit any particular reference is “prior art” to their disclosure.

1. A human carcinoembryonic-antigen-related cell-adhesion molecule 1 (hCEACAM1) protein of SEQ ID NO: 2, comprising one or more amino acid substitutions selected from the group consisting of an R38D substitution, a_V39C substitution, a D40R substitution, a Q89H substitution, a G41S substitution, a V39F substitution, an I91F substitution, or combinations thereof.

2. The hCEACAM1 protein of claim 1, wherein the hCEACAM1 protein comprises an R38D substitution.

3. The hCEACAM1 protein of claim 1, wherein the hCEACAM1 protein comprises a V39C substitution.

4. The hCEACAM1 protein of claim 1, wherein the hCEACAM1 protein comprises a D40R substitution.

5. The hCEACAM1 protein of claim 1, wherein the hCEACAM1 protein comprises a Q89H substitution.

6. The hCEACAM1 protein of claim 1, wherein the hCEACAM1 protein comprises a G41S substitution.

7. The hCEACAM1 protein of claim 1, wherein the hCEACAM1 protein comprises a V39F substitution.

8. The hCEACAM1 protein of claim 1, wherein the hCEACAM1 protein comprises an I91F substitution.

9. The hCEACAM1 protein of claim 1, wherein the hCEACAM1 protein comprises an R38D substitution and an I91F, substitution.

10.-14. (canceled)

15. The hCEACAM1 protein of claim 141, wherein the hCEACAM1 protein is linked to one or more moieties selected from the group consisting of an immunoglobulin Fc region or fragment thereof, an antibody or fragment thereof, an aptamer or fragment thereof, a detectable label or fragment thereof, a ligand or fragment thereof, a therapeutic agent or fragment thereof, a cytotoxic agent or fragment thereof, a receptor or fragment thereof, or combinations thereof.

16. (canceled)

17. (canceled)

18. The hCEACAM1 protein of claim 1, wherein the hCEACAM1 protein comprises one or more non-naturally-occurring amino acids.

19. A method of modulating an immune response for treating a disease or disorder in a subject in need thereof, the method comprising, administering to a subject a pharmaceutical composition comprising a therapeutically effective amount of the hCEACAM1 protein of claim 1.

20. The method of claim 19, wherein the subject has a disease or disorder selected from the group consisting of an autoimmune disease, a cancer, an inflammatory disease, a bacterial infection, a viral infection, or combinations thereof.

21. The method of claim 19, wherein the hCEACAM1 protein comprises an R38D substitution.

22.-26. (canceled)

27. The method of claim 19, wherein the hCEACAM1 protein comprises an I91F substitution.

28. The method of claim 19, wherein the hCEACAM1 protein comprises an R38D substitution and an I91F substitution.

29.-33. (canceled)

34. The method of claim 19, wherein the hCEACAM1 protein is linked to one or more moieties selected from the group consisting of an immunoglobulin Fc region or fragment thereof, an antibody or fragment thereof, an aptamer or fragment thereof, a detectable label or fragment thereof, a ligand or fragment thereof, a therapeutic agent or fragment thereof, a cytotoxic agent or fragment thereof, a receptor or fragment thereof, or combinations thereof.

35.-37. (canceled)

38. A nucleic acid encoding the hCEACAM1 protein of claim 1.

39.-57. (canceled)

58. A vector comprising the nucleic acid of claim 38.

59. A cell comprising the vector of claim 58.


SEQUENCE LISTING (4 sequences)

SEQ ID NO: 1 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 2 - protein, 492 aa, Homo sapiens
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 3 - protein, 142 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYP

SEQ ID NO: 4 - protein, 108 aa, Homo sapiens
QLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYP